(19) United States
(12) Patent Application Publication
(10) Pub. No.: US 2025/0161374 A1
(43) Pub. Date: May 22, 2025

(54) BACILLUS COAGULANS STRAIN, COMPOSITIONS THEREOF, AND METHODS OF USE

(71) Applicant: DEERLAND PROBIOTICS & ENZYMES, INC., Kennesaw, GA (US)

(72) Inventor: John DEATON, Kennesaw, GA (US)

(73) Assignee: DEERLAND PROBIOTICS & ENZYMES, INC., Kennesaw, GA (US)

(21) Appl. No.: 18/712,764

(22) PCT Filed: Dec. 8, 2022
(86) PCT No.: PCT/US2022/081195

§ 371 Date: May 23, 2024

(30) Foreign Application Priority Data
Dec. 8, 2021 (IE) 2021/0210

(51) Int. Cl. A61K 35/742

(52) U.S. Cl. CPC A61K 35/742

(57) ABSTRACT

The invention is directed to new Bacillus coagulans strain, which alone or in combination with other Bacilli strains can be used as probiotics or together with a prebiotic and a symbiotic. The invention also relates to a composition such as a pharmaceutical composition, dairy product, functional food, nutraceutical, dietary supplement, and product for personal care comprising the new Bacillus coagulans strain alone or in combination with other strains, as well as use of the strain for prevention or treatment gastrointestinal infections and diseases, and other uses.

### FIELD OF THE INVENTION

This invention relates to a new Bacillus coagulans strain, which alone or in combination with other Bacilli strains can be used as probiotics or together with a prebiotic and a symbiotic. The invention also relates to a composition such as a pharmaceutical composition, dairy product, functional food, nutraceutical, dietary supplement, and product for personal care comprising the new Bacillus coagulans strain alone or in combination with other strains, as well as use of the strain for prevention or treatment gastrointestinal infections and diseases, and other uses.

### BACKGROUND OF THE INVENTION

Probiotics are live microorganisms or microbial mixtures administered to improve the patient's microbial balance, particularly the environment of the respiratory and gastrointestinal tract. Bacillus strains have been employed for the treatment of respiratory infections, prevention of diarrhoea, as well as, for the treatment of immuno-related diseases (Elshaghabee et al., 2017).

The normal intestinal flora is dominated by various bacterial species, which produce substances that help control the growth of pathogens. Dysbiosis is a condition that is characterized by a decrease of the certain bacterial species and an increased growth of pathogenic bacteria. Dysbiosis has been associated with the development of periodontal disease, inflammatory bowel disease, and chronic fatigue syndrome. Some studies have suggested patients with dysbiosis may have an increased risk of developing metabolic and cardiac disorders (Chan et al., 2013).

By administering probiotic Bacilli, it is possible to regenerate the intestinal flora of men and women with recurrent episodes of dysbiosis. Dysbiosis is a common gastrointestinal problem. Dysbiosis caused by Escherichia coli is also a common problem (Chan et al., 2013).

The presence of Bacilli is important for the maintenance of the intestinal microbial ecosystem. Bacilli have been shown to possess inhibitory activity toward the growth of pathogenic bacteria such as Listeria monocytogenes, Escherichia coli, Salmonella spp. and others (Yilmaz et al., 2005). This inhibition could be due to the production of inhibitory compounds such as organic acids, hydrogen peroxide, bacteriocins or reuterin or to competitive adhesion to the epithelium (Abriouel et al., 2010).

Bacilli have also been examined as a treatment of respiratory tract infections (Marseglia et al., 2007). For example, the installation of Bacilli, and stimulation of indigenous organisms has been employed to prevent recurrence of urinary tract infections (Marseglia et al., 2007). The role of Bacilli in preventing intestinal infections has also been investigated.

### DESCRIPTION OF RELATED ART

The importance of Bacilli as probiotics has been described in the literature.

Hyronimus et al., 2000 discloses the screening of probiotic activities of a number of Bacilli strains by in vitro techniques and evaluation of the colonization ability of thirteen selected strains in humans. The strains were examined for resistance to pH 2.5 and 0.3% Oxgall adhesion to Caco-2 cells and antimicrobial activities against enteric pathogenic bacteria (Khochamit et al., 2015). Bacilli have been shown to possess the primary requirement of GIT stress tolerance, besides having good adhesion and bio-therapeutic properties (Thakur et al., 2016).

Pharmaceutical compositions of Bacilli known in the art are not sufficiently efficient in recolonizing in vivo i.e., mammalian microbial ecosystems and there is, therefore, a need to find Bacilli with an inherent ability to recolonize upon administering the Bacilli in the form of a pharmaceutical composition, a nutraceutical, a dairy product, a functional food or absorbent product. Bacilli isolated from soil, may have the ability to recolonize in vivo upon administration because of their inherent ability to survive in the human microbial ecosystem. It is often a cumbersome process to identify Bacilli strains with enhanced abilities to colonize upon administration and it is therefore important to select the best test systems to predict their in vivo ability to colonize.

In the literature, there seems to be a large variation in the reported in vitro adherence of probiotic strains. This variation indeed reflects biological differences between strains, but certainly also depends on experimental conditions. Moreover, there also seems to be variation with regard to how to measure the adherence. It may be argued that an in vitro experiment only serves as a means to estimate the in vivo ability to colonize by adherence to epithelial cells.

Despite being long considered soil microorganisms, Bacillus spp. have been used for more than 50 years in the form of fermentation products or spore-based supplements (Cutting et al., 2011). Bacilli, being ubiquitous in nature, consistently enter the gastrointestinal and respiratory tracts of healthy people through food, water, and air (Benno & Mitsuoka, 1986). They have been isolated from the gut and can reach up to 10⁷ CFU/g and hence are considered to be one of the dominant components of the normal gut microbiota (Lakshmi et al., 2017). More recently, strains of Bacillus clausii have been isolated in order to provide more specific functions and its safety has been evaluated. Bacillus clausii has been previously used in diarrhoeal patients (Sudha et al., 2013, Horosheva et al., 2014) and children with recurrent respiratory infections (Marseglia et al., 2007) with no adverse events reported. Though the countries and strains are not specified, Bacillus clausii has been commercialized in 55 countries around the world (Nista et al. 2004; Gabrielli et al. 2009). The literature review for Bacillus clausii showed no adverse events related to the probiotic and the worldwide presence of bacteria in different countries supplements the narrative of its safety for human consumption.

Bacillus coagulans has a long history of use in a variety of foods. There have been many strains of Bacillus coagulans that have been widely consumed around the world for decades (Endres et al., 2009). The presence of this bacterium can be found in foods such as yogurts, milk, sauerkraut, kimchi, and other dairy products, all of which contain levels of Bacillus coagulans from 5×10⁹ CFU/g (Sudha et al., 2016) to 9.38×10¹⁰ (Endres et al., 2011). Several Bacillus species have been reported to be common in honeys and include Bacillus megaterium, Bacillus coagulans, Bacillus subtilis, Bacillus licheniformis, and Bacillus pumilus (Alippi, 1995; Alippi et al., 2004; Gilliam, 1979; Gilliam & Valentine, 1976; Snowdon & Cliver, 1996). The origin of this bacterial species for use in probiotics stems from India, where a number of manufacturers produce Bacillus coagulans as a food ingredient for export and relabeling in Europe and the US (Cutting, 2011). In order to assess the safety of Bacillus coagulans, the genome was investigated and it was found that it did not contain any deleterious genes (Salvetti et al., 2016). Due to the nonhazardous nature of Bacillus coagulans, this bacterium has been reported safe by the US Food and Drug Administration (FDA) and the European Union Food Safety Authority (EFSA) and is on the Generally Recognized as Safe (GRAS) and Qualified Presumption of Safety (QPS) list (European Food Safety Authority, 2017). Some of the non-pathogenic strains among the 100 known Bacillus spp., including Bacillus coagulans and Bacillus subtilis, were stated as safe for human consumption for all ages (Nithya & Halami, 2012). A literature review for Bacillus coagulans shows no adverse events related to the probiotic which solidifies the findings that is well tolerated and safe. Therefore, Bacillus coagulans may be considered a probiotic of safe and consumption providing benefit to the host.

Bacillus megaterium has been found on diverse habitats from soil to seawater, sediment, rice paddies, honey, fish, milk and dried foods (Alfoldi, 1957; Alippi & Reynaldi, 2006; Padgham and Sikora, 2007; Pelletier & Sygusch, 1990; Vary et al., 2007; Von Tersch and Carlton, 1983; Scholle et al., 2003, Kotb, 2014). Further qualitative analysis of microorganisms isolated from honeys revealed that one of the most frequent species of Bacillus is Bacillus megaterium (Alippi, 1995; Alippi et al., 2004; Snowdon & Cliver, 1996; Tysset, Durand, & Taliergio, 1970). There have been supplemental studies that have isolated Bacillus megaterium in fish (Sumathi et al., 2017). Afrilasari et al., 2015 also successfully isolated Bacillus megaterium from catfish digestive tract and identified the strain as PTB 1.4. The nonhazardous nature of Bacillus megaterium has landed the bacteria on the Qualified Presumption of Safety (QPS) list (European Food Safety Authority, 2017). Bacillus megaterium strain ATCC 14581 has been confirmed through genome-analysis to be nearly identical (>99%) to Bacillus megaterium MIT411. Health Canada stated the organism is not hazardous to human health or the environment; and exposure to the environment and Canadians is medium. Therefore, it is concluded that Bacillus megaterium strain ATCC 14581 is not harmful to human health or to the environment (Health Canada, 2018).

In summary, Bacilli strains with probiotic capabilities should be able to adhere to other suitable cells, such as the cell line Caco-2 cells. Moreover, it is also desirable that the Bacilli strains with probiotic capabilities show in vitro inhibitory activity against other bacterial species, produce acid after growth in liquid culture and/or produce hydrogen peroxide.

### SUMMARY OF THE INVENTION

It is an object of the present invention to provide strains and compositions as described throughout this application such as pharmaceutical formulations or absorbent products of suitable probiotic Bacilli strains with the desired properties. In an embodiment, the present invention concerns the Bacillus coagulans strain CGI314 alone or in combination with other strains such as Bacilli strains such as Bacillus megaterium strain MIT411 (disclosed and claimed in corresponding PCT Application PCT/US2022/xxxxx claiming priority from Irish Patent Application No. 2021/0211, whose contents are incorporated herein in their entirety) and Bacillus clausii strain CSI08 (disclosed and claimed in corresponding PCT Application PCT/US2022/xxxxx claiming priority from Irish Patent Application No. 2021/0209, whose contents are incorporated herein in their entirety). In an embodiment, these strains have similar or essentially the same advantageous properties e.g. the ability to colonize by adherence to mucosal membranes and which are therefore suited for the treatment or prevention of infections or diseases for instance such as the vaginal, urinary-tract, gastrointestinal, naso-sinal, pharyngeal, esophageal, oral, and/or other areas of the body with e.g. mucosal membranes, as well as, treatment or prevention of infections or diseases of the skin and/or other areas of the body having epithelium; immune health, protection against oxidative stress, cleansing and detoxification, metabolic health and cardiovascular health amongst others. such as providing antimicrobial activity including lactic acid, benzoic acid, and succinic acid, anti-inflammatory activity, suppression of pro-inflammatory response, activating and/or provoking immune response eg. by stimulating macrophages, providing immunoprotection, aiding in digestion and/or fermentation for instance in the gut, producing branched amino acides, essential amino acids and group B vitamins, maintaining healthy gut and/or skin including lactic acid benzoic acid, and succinic acid in support of skin health, protection of mucosal and other epithelial tissues from toxic agents, decreasing incidence of loose stools, improving the gut-brain axis, and treating and/or preventing dysbiosis and its effects such as periodontal disease, inflammatory bowel disease, chronic fatigue syndrome, metabolic disorders, cardiac disorders, respiratory trat infections, urinary tract infections, GI infections, and diarrhea; and restoring normal and/or healthy flora. In an embodiment, the present invention allows the use of Bacillus clausii strain CSI08 and compositions for use in fecal transplants.

Gastrointestinal diseases include, but are not limited to treating gastrointestinal irregularity in an individual, wherein the individual has at least one 24-hour episode per month of bowel movements measuring 1 or 2 on the Bristol Stool Scale (i.e. treating constipation; or wherein the individual has at least one 24-hour episode per month of bowel movements measuring 6 to 7 on the Bristol Stool Scale (tending towards diarrhoea), wherein the frequency of the individual's 24-hour episodes per month of bowel movements measuring 1 or 2 (or 6 to 7) on the Bristol Stool Scale decreases.

Also included is a method of restoring gastrointestinal regularity in an individual, wherein the individual has at least one 24-hour episode per month of bowel movements measuring 1 or 2; or 6 to 7 on the Bristol Stool Scale, wherein the frequency of 24-hour periods of the individual's bowel movements measuring from 3 to 5 on the Bristol Stool Scale increases.

The invention further includes maintaining healthy gut microflora, with Bacillus-containing composition(s). The Bacillus-containing composition(s) can be used as probiotic supplementation of the gastrointestinal microflora, and may compete with or otherwise discourage pathogenic bacteria in the gut such as Escherichia coli, Listeria monocytogenes, Salmonella spp.

Another object of the present invention is to provide pharmaceutical formulations with an increased ability to colonize by adherence to the mucosal membrane by employing mucous adhesive excipients.

It is a further object of the present invention to provide vaginal formulations with an increased ability to suppress the growth of Candida albicans and Gram-negative pathogenic bacteria.

It is yet another objective of the present invention to provide compositions such as dairy products, nutraceutical products and functional foods comprising Bacillus coagulans strain CGI314 alone or combination with other Bacilli strains such as a Bacillus megaterium strain and/or a Bacillus clausii strain, having essentially the same properties having the ability to colonize the mucosal membranes and therefore adapted to treatment or prevention of vaginal infections, urinary-tract infections and gastrointestinal diseases. Compositions of the present invention may be administered for 1 dose, 1 day, 1 day to 1 week, 1 day to 1 month, 1 month to 45 days, 45 days to 2 months, 3 months, 6 months, 1 year, or more, including any timeframe identified and/or falling within these ranges.

The present invention includes methods of treatment and/or preventions and various other methods, which may comprise the steps of providing a Bacillus coagulans strain or composition of this invention to a subject (for instance a mammal subject, a human including a human patient, and the like), and administering the strain or composition to the subject. In an embodiment, the present invention is directed to a composition, for instance use of a composition, to treat a disease or infection or other condition. In an embodiment, the present invention is directed to the use of a Bacillus coagulans strain or composition thereof, as described throughout this application (including its claims), in the manufacture of a medicament for the treatment of vaginal infections, urinary tract infections, gastrointestinal diseases, improving immune health, protection against oxidative stress, cleansing and detoxification, metabolic health, cardiovascular health, and/or skin health, and/or other treatments or other reasons for application described throughout this application.

### FIGURES

In the drawings:

FIG. 1 illustrates the phylogenetic tree (16S) of Bacillus spp, arranged in clades.

FIG. 2 illustrates the phylogenetic tree (gyrB) of Bacillus spp., arranged in clades.

FIG. 3 shows antimicrobial activity of B. coagulans CGI314 against gut, skin and urinary tract opportunistic pathogens on MRS agar plates with 0.4% TSA agar overlays (solid media). A—E. coli; B—S. enteritidis and C—S. aureus.

FIG. 4 shows B. coagulans CGI314 antimicrobial activity in liquid TSB media against gut, skin and urinary tract opportunistic pathogens. Pathogens: E. coli, Salmonella, Pseudomonas aeruginosa and S. aureus. (Concentration Log₁₀ CFU/ml)

FIG. 5 shows the strongest antimicrobial activity observed with B. coagulans CGI314 (Fortispore) against E. coli at 24 hr. (a) B. coagulans MTCC5856 (Lactospore®) (b) B. coagulans CG314 (Fortispore) (c) B. coagulans 6086 (BC30™) inoculated on MRS agar against E. coli 0.4% TSA agar overlay plates.

FIG. 6 shows the strongest antimicrobial activity observed with Fortispore against S. enteritidis at 48 hr. (a) Lactospore® (b) Fortispore (c) BC30™ inoculated on MRS agar against S. enteritidis 0.4% TSA agar overlay plates.

FIG. 7 shows no antimicrobial activity was detected across Bacillus coagulans against P. aeruginosa (a) B. coagulans MTCC5856 (Lactospore®) (b) B. coagulans CG314 (c) B. coagulans 6086 (BC30™) inoculated on TSA agar against P. aeruginosa 0.4% TSA agar overlay plates at 24 hr.

FIG. 8 shows limited antimicrobial detected across B. coagulans against S. aureus using MRS agar at 24 hr. (a) B. coagulans inoculated on TSA agar against S. aureus 0.4% TSA agar overlay plates. (b) B. coagulans inoculated on MRS agar against S. aureus 0.4% TSA agar overlay plates.

FIG. 9 shows total antioxidant capacity of B. coagulans (Fortispore) and L. rhamnosus.

FIG. 10 shows total antioxidant capacity of DE111, B. coagulans (Fortispore), B. clausii CSI08 (Munispore), B. megaterium MIT411 (Renuspore) and L. rhamnosus.

FIG. 11 shows adherence of Fortispore to the HT-29 cell line, measured as percentage of adherence bacteria, was negligible.

FIG. 12 shows adherence of Fortispore to the HT-29-MTX cell line, measured as percentage of adherence bacteria, is negligible.

FIG. 13 shows a study on the adherence of B. coagulans strains to the HT-29 cell line.

FIG. 14 show a study on the adherence of B. coagulans strains to the HT-29-MTX cell line.

FIG. 15 shows the absence of caseolytic activity in B. coagulans CGI314 using both streak and overnight TSB broth method at 24 h or 48 h.

FIG. 16 shows proteolytic activity of Fortispore towards casein derivatives is lower than positive control Proteinase K using EnzCheck® kit following incubation at 37° C. for 24 h.

FIG. 17 shows absence of caseolytic activity across (left to right) B. coagulans CGI314, 20 B. coagulans 6086 (BC30™) and B. coagulans MTCC5856 (Lactospore®) using both streak and overnight TSB broth method at 24 h or 48 h.

FIG. 18 shows Fortispore B. coagulans showing low protease activity towards casein.

FIG. 19 shows a quantitative analysis of the caseolytic activity across B. coagulans strains determined by EnzCheck® kit following incubation at 37° C. for 24 h.

FIG. 20 shows FAA profile of Fortispore UTH fermented milk. Relative concentrations of Methionine, Alanine, Proline, Tryptophan, Lysine, cis-Aconitic acid are shown (white bar (left) represents Control).

FIG. 21 shows FAA profile of Fortispore UTH fermented milk. Relative concentrations of Succinic acid, Lactic acid, Benzoic acid, Isocitric acid are shown (white bar (left) represents Control).

FIG. 22 compares Fortispore with Lactospore and BC30 in the production of Lactic acid, Succinic acid, and Benzoic acid (left to right: Control, BC30, Lactospore, Fortispore).

FIG. 23 compares Fortispore with Lactospore and BC30 in the production of amino acids (left to right: Control, BC30, Lactospore, Fortispore; also, showing relative concentrations of Methionine, Proline, Tryptophan, Lysine).

FIG. 24 shows Fibersol® (F) significantly increased the concentration (CFU/mL) of Fortispore by 1 log₁₀ in minimal media 24 hours post incubation compared to controls.

FIG. 25 shows Fibersol® (F) significantly increases the concentration (CFU/mL) of Fortispore in Minimal media whereas no significance in the growth of BC30 and Lactospore were seen.

FIG. 26 shows that Fibersol® (F) did not show significant increase in the concentration (CFU/mL) of Fortispore in TSB media compared to controls.

FIG. 27 shows Fibersol® (F) did not show significant increase in the concentration (CFU/mL) of DE111, Fortispore, BC30, E. coli and Salmonella enteritidis in TSB media compared to controls.

FIG. 28 shows Fibersol® (F) did not significantly increase the concentration (CFU/mL) of Fortispore in 50% TSB media compared to controls.

FIG. 29 shows Fibersol® (F) significantly increased the yield (CFU/mL) of DE111 by 1 log₁₀ after 24 hours in 50% TSB media.

FIG. 30 shows that Fibersol® (F) did not show significant increase in the concentration (CFU/mL) of Fortispore compared to controls in BHI media.

FIG. 31 shows that Fibersol® (F) did not show significant increase in the concentration (CFU/mL) of DE111, Fortispore and BC30 compared to controls in BHI media.

FIG. 32 shows that Fibersol® (F) did not show significant increase in the concentration (CFU/mL) of Fortispore compared to controls in 50% BHI media.

FIG. 33 shows that Fibersol® (F) did not show significant increase in the concentration (CFU/mL) of DE111, Fortispore and BC30 compared to controls in 50% BHI media.

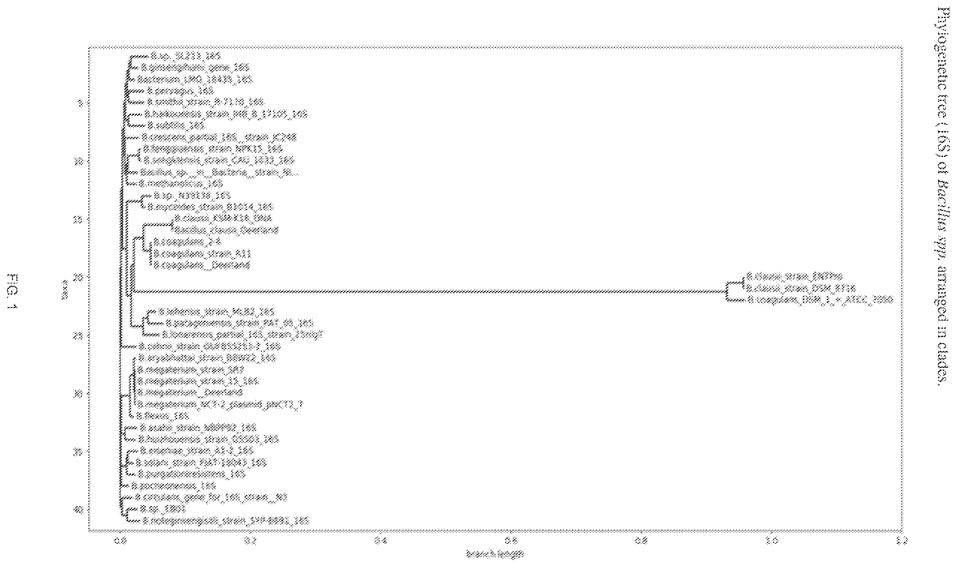

### DETAILED DESCRIPTION OF THE INVENTION

### Genotypic Identification

The Applicant collaborated with Cornell University (Ithaca NY, USA) for genomic sequencing and identification.

### WGS DNA Composition

The whole genome sequence (WGS) was carried out by Cornell University, including assembly and annotation. Bioinformatics analysis was completed at Cornell University and at Deerlands Probiotics and Enzymes (Kennesaw, GA, USA). Identifying gyrB gene poymorphism was carried out the the Applicant.

The gyrB gene encodes DNA gyrase subunit B. DNA gyrase negatively supercoils closed circular double-stranded DNA in an ATP-dependent manner to maintain chromosomes in an underwound state. Gene sequencing analysis used the gyrB gene polymorphism, a well-established method for species level discrimination of prokaryotes (Bavykin et al., 2014; Wang et al., 2007). The representative genomes ere reviewed and curated by NCBI, and coordinated with the UniProtein Consortium (NCBI, 2016; UniProt, 2016). R package SequinR coupled with the UniProt Consortium analysis was used to compare whole genome sequences (WGS) and GyrB sequence of the presently claimed Bacillus coagulans strain CGI314 with other reference strains (Tables 1 to 3 below)

Genotypic, gyrB, & 16S rRNA Identification of Bacillus coagulans CGI314

CGI314 was isolated, and the genome was considered successful.

The genome size (3.0 Mbp) of CGI314 was shorter than that of previously sequenced B. coagulans strain (3.4 Mbp) (Upadrasta et al., 2016). The % GC (47.3%) is consistent with that of a previously sequenced B. coagulans strain (46.5%) (Upadrasta et al., 2016).

TABLE 1
Whole genome sequencing metrics of CGI314

| | Number of | Total | GC | N50 | Average |
|---|---|---|---|---|---|
| Strain | contigs | length (nt) | (%) | (nt) | Coverage (x) |
| Bacillus | 210 | 3,028,595 | 47.30 | 29,700 | 71 |
| coagulans | | | | | |
| CGI314 | | | | | |

TABLE 2
Distance matrix of gyrB gene

| Bacillus | | | | |
|---|---|---|---|---|
| coagulans subsp. | ATCC 7050 | BC-30 | IS-2 | MTCC 5856 |
| CGI314 | 99.9% | 95.2% | 95.2% | 95.2% |

TABLE 3
Whole genome sequence comparison

| Bacillus | | | Sequence |
|---|---|---|---|
| coagulans subsp. | Accession No. | % GC | Length |
| CGI314 | JABBFU000000000.1 | 47.30% | 3,028,595 |
| ATCC 7050 | NZ_CP009709.1 | 46.90% | 3,366,995 |
| BC-30 | JPSK01000000 | 46.39% | 3,458,616 |
| IS-2 | JZDH01000000 | 46.41% | 3,446,692 |
| MTCC 5856 | NZ_CP011939.1 | 46.23% | 3,694,837 |

16S rRNA

Whole genome sequencing (WGS) and 16S rRNA analysis of CGI314, as compared to the four reference strains, exhibited an average nucleotide identity (ANI) score for 16S rRNA of 99.9% when compared to the B. coagulans strain ATCC 7050. The genome size (3.0 Mbp) and GC content (47.30%) for CGI314 was comparable to the four reference strains.

### Further Deposits and Accession Numbers

Genome sequence data of Bacillus coagulans strain CGI314 (Fortispore) was deposited into NCBI GenBank database, and the genome sequence was annotated with the NCBI Prokaryotic Genome Annotation Pipeline (PGAP). The genome is publicly available, with GenBank Accession Number JABBFU000000000.1 for the strain, and available for instance at the link: https://www.ncbi.nlm.nih.gov/nuccore/JABBFU000000000.1.

Genome sequence data of Bacillus clausii strain CSI08 (Munispore) was deposited into NCBI GenBank database, and the genome sequence was annotated with the NCBI Prokaryotic Genome Annotation Pipeline (PGAP). The genome is publicly available, with GenBank Accession Number JABBNL000000000.1 for the strain, and available for instance at the link: Alkalihalobacillus clausii strain CSI08, whole genome shotgun sequenci—Nucleotide—NCBI (nih.gov).

Genome sequence data of Bacillus megaterium strain MIT411 (Renuspore) was deposited into NCBI GenBank database, and the genome sequence was annotated with the NCBI Prokaryotic Genome Annotation Pipeline (PGAP). The genome is publicly available, with GenBank Accession Number JABBNK000000000.1 for the strain, and available for instance at the link: Priestia megaterium strain MIT411, whole genome shotgun sequencing pro—Nucleotide—NCBI (nih.gov).

Phylogenetic Placement, carried out by Deerland Probiotics and Enzymes, Inc.

Genome-to-genome distance calculation (GGDC), a digital gold standard, is as reliable as DNA-DNA hybridization (DDH) (Auch et al. 2010). GGDC holds more discriminatory power for subspecies delineation and subsequently, was used as a confirmation of multiple alignment and phylogenetic analyses. GGDC yielded calculation-based models that further verified that Bacillus coagulans CGI314 is a close relative to ATCC 7050.

Although the conserved 16S rRNA sequence is a well-established method to compare and study phylogenies in bacteria, the high proportion of sequence similarity between closely related species limits its usefulness (Wang et al., 2007). High rates of 16S rRNA sequence similarity in closely related bacterial species are due to a slower rate of molecular evolution. Past research (Bavtlin et al., 2004; Wang et al., 2007) supports the validity of using gyrB sequences as taxomonic biomarkers due to their rate of base substitutions and significant and reliable correlation with DNA-DNA Hybridization analysis (Dauga et al., 2002; Kasai et al., 1998; Wang et al., 2007). The gyrB encodes DNA gyrase B, and type II topoisomerase that plays an important role in DNA replication. Gyrase B subunits are encoded by the gyrB gene.

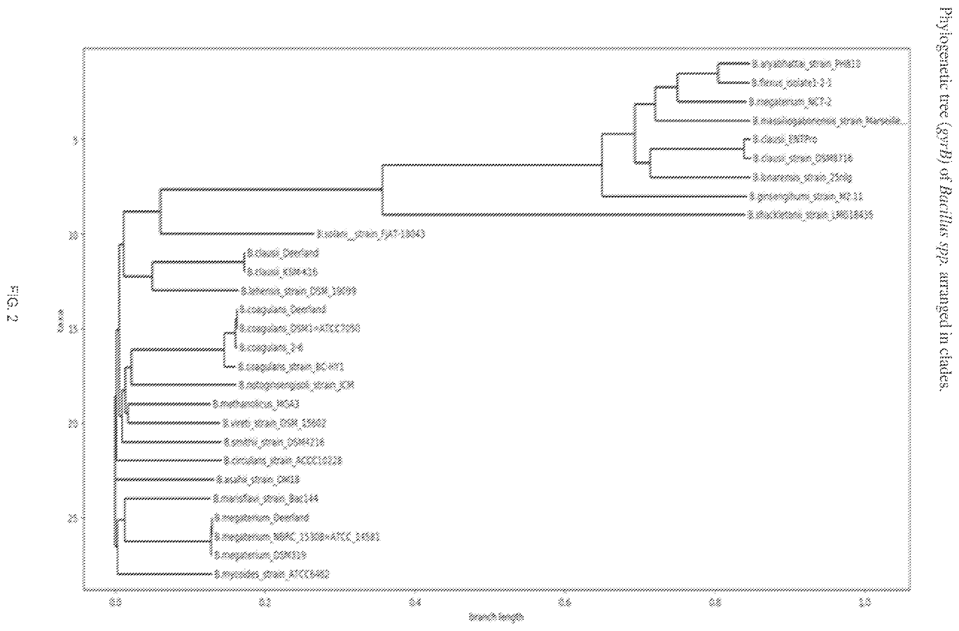

Phylogenetic analysis using neighbor-joining (NJ) method (Saitou & Nei, 1987) placed Bacillus coagulans CGI314 in a clade with Bacillus coagulans ATCC 7050 (FIG. 2). This confirms all previous genomic identity determinations. Bacillus coagulans CGI314 has been placed in the Bacillus coagulans group.

### Definitions

By “excipient” is meant any non-active ingredient that is added to form part of the final formulation.

By “probiotic” is meant a viable microbial supplement, which has a beneficial influence on the patient through its effects in the intestinal tract, urinary tract or the vaginal tract. The term “probiotic(s)” can refer to live microorganisms which, when administered in adequate amounts, confer a health benefit on the host. Foods and food additives containing probiotics may support the restoration of the healthy balance of the gut microflora. Further, probiotic supplementation of the intestinal flora may promote healthy intestinal homeostasis.

A “prebiotic” is used herein as a substrate, which has a beneficial effect on a probiotic and thus on the individual patient taking the probiotic. Suitable prebiotics may be selected from an inulin, an oligosaccharide, and/or a vitamin.

A “subject” is used herein includes a person suffering from any clinical condition related to a microbial imbalance as well as a person using bacterial preparations prophylactically. Optionally, the subject is a human.

By a “symbiotic product” is meant a combination of probiotic and prebiotic, which is synergy, have a beneficial influence on the patient.

By “hardy growth” is meant that bacteria show excellent growth.

The abbreviation “CFU” means colony forming units.

The present invention relating to a probiotic Bacilli strain capable of regenerating the in vivo flora in subjects will become apparent in the progress of the following detailed description.

According to a first aspect, the present invention comprises Bacillus coagulans CGI314 alone or in combination with other probiotic Bacilli strains with essentially the same properties. Such other probiotic Bacilli stains may include, but are not limited to a Bacillus clausii strain and a Bacillus megaterium strain. Such other Bacilli strains may further include a Bacillus clausii strain and a Bacillus megaterium strain each filed today under these respective titles—their contents are incorporated herein in their entirety.

SEQ ID NO: 1, as recited in the claims attached hereto, comprises gyrB of Bacillus coagulans CGI314.

SEQ ID NO: 2, as recited in the claims attached hereto, comprises 16S rRNA of Bacillus coagulans CGI314.

SEQ ID NO: 3, as recited in the claims attached hereto, comprises the assembled whole genome sequence of Bacillus coagulans CGI314.

The Bacillus strain claimed herein, with reference to at least 97% identity to SEQ ID NO: 1 and/or 2; or to at least 97% identity to SEQ ID NO: 3, has the following properties:

Bacillus coagulans CGI314

The strain shows bile stability.

The strain shows acid stability.

The strain show heat tolerance.

The strain produces a natural antibiotic substance in the form of a bacteriocin.

In order to determine the genus and species of the strains disclosed herein, the whole genome was sequenced. The amount and composition of the strains were identified and determined.

The strain was shown to possess little to no antibiotic resistance and no safety concerns.

The strain was found to show stability toward acid and bile.

According to a second aspect, the Bacilli strain of the present invention is suitable for medical use in preventing or treating vaginal infections, urinary tract infections and gastrointestinal diseases (including gastrointestinal infections), as well as, improving immune health, protection against oxidative stress, cleansing and detoxification, metabolic health and cardiovascular health.

In another preferred embodiment, a pharmaceutical composition is provided comprising Bacillus coagulans CGI314 alone or in combination with other probiotic Bacilli strains with essentially the same properties, together with a pharmaceutically acceptable carrier and/or diluent. Such other probiotic Bacilli stains include, but are not limited to a Bacillus clausii strain and a Bacillus megaterium strain. The bacterial strains are formulated into pharmaceutical formulations in order to allow the easy administration of the probiotic strains and by means known to the man skilled in the art.

Bacillus coagulans has been proven able to alleviate symptoms of irritable bowel syndrome (Sudha et al., 2018), improve muscle integrity and cytokine response (Gepner et al., 2017; Jager et al., 2018), modulate the gut microbiome and the immune response (Kimmel et al., 2010), reduce function intestinal gas symptoms (Kalman et al., 2009), reduce the instance and duration of diarrhea (Dolin et al., 2009), improve the symptoms of functional abdominal pain and bloating (Hun et al., 2009), protect against acetaminophen induced acute liver injury (Neag et al., 2020), enhance butyrogenesis (Sasaki et al., 2020), reduce severity of bacterial vaginosis (Sudha et al., 2012), and reduce cholesterol (Sudha et al., 2012) all in vivo. Bacillus coagulans has also shown to induce immune response and anti-inflammatory action (Jensen et al., 2017), improve plant protein digestion (Keller et al., 2017), adhere to Caco-2 cells (Sharma & Kanwar, 2017), improve colonic microenvironment in patients with ulcerative colitis (Sasaki et al., 2020), reduce the adhesion, cytotoxicity and induction of apoptosis caused by S. typhimurium in HT-29 cells (Kawarizadeh et al., 2019), hydrolyze lactose from whey protein (Liu et al., 2019), and enhancing t-cell response (Baron, 2009) all in vitro.

Bacillus clausii has been proven efficacious in preventing recurrent respiratory infections (Marseglia et al., 2007), reducing duration and severity of diarrhoea (Sudha et al., 2019) in vivo. Bacillus clausii has also been proven capable to produce protein hydrolysates with antimicrobial and antioxidant capacity (Rochin-Medina et al., 2017), protect against acetaminophen induced acute liver injury (Neag et al., 2020), inhibit cytotoxic effects induced by Clostridium difficile and Bacillus cereus toxins (Ripert et al., 2016) in vitro.

Bacillus megaterium has been shown to exert protective effects against oxidative stress both in vitro and in vivo (Mazzoli et al., 2019). Bacillus megaterium has also been shown capable of adapting and surviving in acid stress conditions and chelating heavy metals in vitro (Ferreira et al., 2019).

Preferably, the probiotic bacteria employed in a pharmaceutical in accordance with the present invention are used in bacterial concentration of 10⁶-10¹³. In an embodiment, the probiotic bacteria employed in this invention are used in bacterial concentration of 10⁶-10¹³ CFU (colony forming units), for instance as a daily dose, including any amount or range that is included in said range. In an embodiment, the bacteria are employed in an amount of 10⁷-10¹² CFU, or 108-10¹¹ CFU, or 10⁹-10¹⁰ CFU, or for instance in an amount of about 10⁶, about 10⁷, about 108, about 10⁹, about 10¹⁰, about 10¹¹, about 10¹², and/or about 10¹³ CFU, and any amount or range including or between said amounts. In an embodiment, a composition of this invention comprises, consists essentially of, consists of, and/or is characterized by about 10⁶- about 10¹³ CFU such as about 10⁹ Bacillus coagulans CGI314. In an embodiment, a composition of this invention comprises Bacillus coagulans CGI314 (for instance about 10⁹ CFU) in combination with Bacillus megaterium MIT411 and/or Bacillus clausii CSI08. In an embodiment, a composition of this invention is orally administered in capsule form. In an embodiment, in a composition and/or use or method of this invention, Bacillus coagulans CGI314 is in spore form, or is not in spore form.

In certain embodiments, compositions comprising Bacillus coagulans CGI314 can include one or more dry carriers selected from the group consisting of trehalose, maltodextrin, rice flour, microcrystalline cellulose, magnesium stearate, inositol, fructooligosaccharide, galactooligosaccharide, dextrose, dried dairy products, and the like. In certain embodiments, the dry carrier can be added to the compositions comprising Bacillus coagulans CGI314 in a weight percentage of from about 1% to about 95% by weight of the composition.

In certain embodiments, the compositions comprising Bacillus coagulans CGI314 can include one or more liquid or gel-based carriers, selected from the group consisting of water and physiological salt solutions, urea, alcohols and derivatives thereof (e.g., methanol, ethanol, propanol, butanol), glycols (e.g., ethylene glycol, propylene glycol), and the like; natural or synthetic flavorings and food-quality coloring agents, all compatible with the organism; thickening agents selected from the group consisting of corn starch, guar gum, xanthan gum, and the like; one or more spore germination inhibitors selected from the group consisting of hyper-saline carriers, methylparaben, guargum, polysorbate, preservatives, and the like. In certain embodiments, the one or more liquid or gel-based carrier(s) can be added to the compositions comprising Bacillus coagulans CGI314 in a weight/volume percentage of from about 0.6% to about 95% weight/volume of the composition. In certain embodiments, the natural or synthetic flavoring(s) can be added to the compositions comprising Bacillus coagulans CGI314 in a weight/volume percentage of from about 3.0% to about 10.0% weight/volume of the composition. In certain embodiments, the coloring agent(s) can be added to the compositions comprising Bacillus coagulans CGI314 in a weight/volume percentage of from about 1.0% to about 10.0% weight/volume of the composition. In certain embodiments, the thickening agent(s) can be added to the compositions comprising Bacillus coagulans CGI314 in a weight/volume percentage of about 2% weight/volume of the composition. In certain embodiments, the one or more spore germination inhibitors can be added to the compositions comprising Bacillus coagulans CGI314 in a weight/volume percentage of about 1% weight/volume of the composition.

### Delivery System

Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like. Suitable dosage forms include tablets, capsules, solutions, suspensions, powders, gums, and confectionaries. Sublingual delivery systems include, but are not limited to, dissolvable tabs under and on the tongue, liquid drops, and beverages. Edible films, hydrophilic polymers, oral dissolvable films, or oral dissolvable strips can be used. Other useful delivery systems comprise oral or nasal sprays or inhalers, and the like.

For oral administration, probiotics may be further combined with one or more solid inactive ingredients for the preparation of tablets, capsules, pills, powders, granules, or other suitable dosage forms. For example, the active agent may be combined with at least one excipient selected from the group consisting of fillers, binders, humectants, distintegrating agents, solution retarders, absorption accelerators, wetting agents, absorbents, and lubricating agents. Other useful excipients include, but are not limited to, magnesium stearate, calcium stearate, mannitol, xylitol, sweeteners, starch, carboxymethylcellulose, microcrystalline cellulose, silica, gelatin, silicon dioxide, and the like

In certain embodiments, the components of compositions administered according to the methods of the present disclosure, together with one or more conventional adjuvants, carriers, or diluents, may thus be placed into the form of pharmaceutical compositions and unit dosages thereof. Such forms include: solids, and in particular, tablets, filled capsules, powder and pellet forms; liquids, and in particular, aqueous or non-aqueous solutions, suspensions, emulsions, elixirs; and capsules filled with the same; all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The components of the compositions administered according to the methods of the present disclosure can be administered in a wide variety of oral and parenteral dosage forms. It will be obvious to those skilled in the art that the following dosage forms may comprise, in certain embodiments, as the active component, either a chemical compound of the present disclosure or a pharmaceutically acceptable salt of a chemical compound of the present disclosure.

For preparing pharmaceutical compositions to be administered according to the methods of the present disclosure, pharmaceutically acceptable carriers can be either solid or liquid. Solid form preparations include powders, tablets, pills, capsules, cachets, suppositories, and dispersible granules. A solid carrier can be one or more substances that may also act as diluents, flavoring agents, solubilizers, lubricants, suspending agents, binders, preservatives, tablet disintegrating agents, or encapsulating materials.

In powders, the carrier is a finely divided solid, which is in a mixture with the finely divided active component. In tablets, the active component is mixed with the carrier having the necessary binding capacity in suitable proportions and compacted in the shape and size desired.

In certain embodiments, powders and tablets administered according to methods of the present disclosure preferably may contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without additional carriers, is surrounded by a carrier, which is thus in association with it. Similarly, cachets and lozenges are included. Tablets, powders, capsules, pills, cachets, and lozenges are included. Tablets, powders, capsules, pills, cachets, and lozenges can be used as solid forms suitable for oral administration.

Liquid preparations include, but are not limited to, solutions, suspensions, and emulsions, for example, water or water-propylene glycol solutions. For example, parenteral injection liquid preparations can be formulated as solutions in aqueous polyethylene glycol solution. In certain embodiments, chemical compounds administered according to methods of the present disclosure may thus be formulated for parenteral administration (e.g., by injection, for example, bolus injection or continuous infusion) and may be presented in unit dose for administration in ampoules, pre-filled syringes, small-volume infusion, or in multi-dose containers with an added preservative.

The compositions may take such forms as suspensions, solutions, or emulsions in oily or aqueous vehicles, and may contain formulation agents such as suspending, stabilizing, and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

Aqueous solutions suitable for oral use can be prepared by dissolving the active component in water and adding suitable colorants, flavors, stabilizing and thickening agents, as desired. Aqueous suspensions suitable for oral use can be made by dispersing the finely divided active component in water with viscous material, such as natural or synthetic gums, resins, methylcellulose, sodium carboxymethylcellulose, or other well-known suspending agents.

Compositions suitable for topical administration in the mouth include, but are not limited to: lozenges comprising the active agent in a flavored base, usually sucrose and acacia or tragacanth; pastilles comprising the active ingredient in an inert base such as gelatin and glycerine or sucrose and acacia; and mouthwashes comprising the active ingredient in suitable liquid carrier.

Solutions or suspensions are applied directly to the nasal cavity by conventional means, for example, with a dropper, pipette, or spray. The compositions may be provided in single or multi-dose form. In compositions intended for administration to the respiratory tract, including intranasal compositions, the compound will generally have a small particle size, for example, of the order of 5 microns or less. Such a particle size may be obtained by means known in the art, for example, by micronization.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself; or it can be the appropriate number of any of these in packaged form.

Tablets, capsules, and lozenges for oral administration and liquids for oral use are preferred compositions. Solutions or suspensions for application to the nasal cavity or to the respiratory tract are preferred compositions. Transdermal patches for topical administration to the epidermis are preferred.

Further details on techniques for formulation and administration may be found in the latest edition of REMINGTON'S PHARMACEUTICAL SCIENCES (Mack Publishing Co., Easton, PA).

In certain embodiments, compositions of the present invention including compositions administered according to the methods of the present disclosure may also include one or more excipients, most preferably one or more nutraceutical or pharmaceutical excipients. Compositions containing one or more excipients and incorporating one or more probiotics can be prepared by procedures known in the art. Optionally, compositions can include one or more adjuvants, excipients, carriers, buffers, diluents, and/or other customary pharmaceutical auxiliaries. For example, probiotics can be formulated into tablets, capsules, powders, suspensions, solutions for oral administration, solutions for parenteral administration including intravenous, intradermal, intramuscular, and subcutaneous administration, and solutions for application onto patches for transdermal application with common and conventional barriers, binders, diluents, and excipients.

In certain embodiments, nutraceutical compositions including nutraceutical compositions administered according to the methods of the present disclosure may include and may be administered in combination with a pharmaceutically acceptable carrier. In certain embodiments, the active ingredients in such formulations may comprise from about 1% by weight to about 99% by weight. In other embodiments, the active ingredients in such formulations may comprise from about 0.1% by weight to about 99.9% by weight. “Pharmaceutically acceptable carrier” means any carrier, diluent, or excipient that is compatible with the other ingredients of the formulation and not deleterious to the user. Useful excipients include, but are not limited to, microcrystalline cellulose, magnesium stearate, calcium stearate, any acceptable sugar (e.g., mannitol, xylitol), and the like, and for cosmetic use, a water or an oil base may be used, or mixture thereof including such as an emulsion.

### Routes of Administration

The strain Bacillus coagulans CGI314 or a composition comprising a strain of the present invention may be administered by any route, including, but not limited to, oral, sublingual, buccal, ocular, pulmonary, rectal, vaginal, urethral, ureteral, and parenteral administration, or as an oral or nasal spray (e.g., inhalation of nebulized vapors, droplets, or solid particles). Parenteral administration includes, for example, intravenous, intramuscular, intraarterial, intraperitoneal, intranasal, intravaginal, intravesical (e.g., to the bladder), intradermal, transdermal, topical, or subcutaneous administration. Also contemplated within the scope of the invention is the instillation of a pharmaceutical composition in the body of the patient in a controlled formulation, with systemic or local release of the drug to occur at a later time. For example, the drug may be localized in a depot for controlled release to the circulation, or for release to a local site.

Pharmaceutical compositions of the invention may be those suitable for, and formulated for, any of the routes identified above, including for instance oral, rectal, bronchial, nasal, pulmonal, topical (including buccal and sub-lingual), transdermal, vaginal, urethral, ureteral, or parenteral (including cutaneous, subcutaneous, intramuscular, intraperitoneal, intravenous, intraarterial, intracerebral, intraocular injection, or influsion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in the form of shaped articles, e.g., films or microcapsules.

The embodiments described above may be further understood in connection with the following Examples. In addition, the following non-limiting examples are provided to illustrate 5 the invention. However, the person skilled in the art will appreciate that it may be necessary to vary the procedures for any given embodiment of the invention, e.g., vary the order or steps.

### Example 1

### Characterisation of Bacillus coagulans CGI314 (Referred to as Fortispore Hereinafter)

B. coagulans CGI314 (Fortispore) had significant antimicrobial activity against E. coli, Salmonella and S. aureus in solid MRS agar overlayed with 0.4% TSA agar.

However, no antimicrobial activity was observed against P. aeruginosa in solid media. Fortispore has antimicrobial activity against gut, urinary and skin pathogens in solid environments.

TABLE 4
Summary of B. coagulans CGI314 antimicrobial activity against
gut, skin and urinary tract opportunistic pathogens (solid media)

| Probiotic | Zone of inhibition diameter (mm) | | | |
|---|---|---|---|---|
| strain | E. coli | S. enteritidis | S. aureus | P. aeruginosa |
| Fortispore | 28.60 ± 4.10 | 33.40 ± 4.28 | 20.67 ± 0.58 | — |
| CGI314 | | | | |

Antimicrobial activity is indicated as zone of inhibition (mm)±standard deviation.

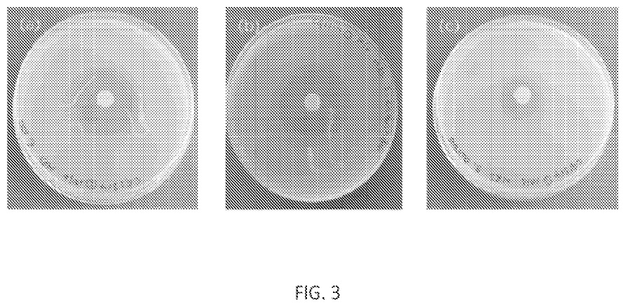

FIG. 3 shows antimicrobial activity of B. coagulans CGI314 against gut, skin and urinary tract opportunistic pathogens on MRS agar plates with 0.4% TSA agar overlays (solid media). A—E. coli; B—S. enteritidis and C—S. aureus.

Fortispore had significant antimicrobial activity against E. coli, Salmonella and P. aeruginosa in liquid TSB media. However, no antimicrobial activity was observed against S. aureus in liquid TSB media.

TABLE 5
Summary of Fortispore antimicrobial activity against gut, skin
and urinary tract opportunistic pathogens (liquid media)

| | Inhibition capacity | | | |
|---|---|---|---|---|
| Conditions | E. coli | S. enteritidis | S. aureus | P. aeruginosa |
| Solid media | + | + | + | − |
| Liquid media | + | + | − | + |

Antimicrobial activity detected (+), no antimicrobial activity observed (−).

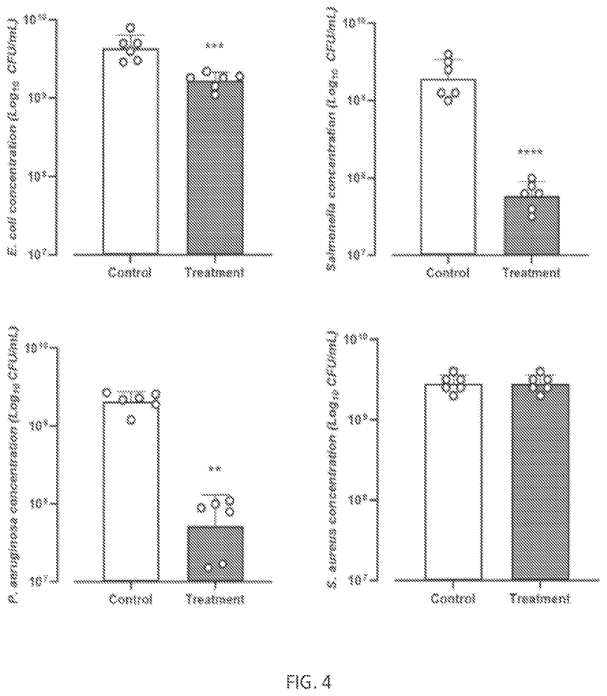

FIG. 4 shows B. coagulans CGI314 antimicrobial activity in liquid TSB media against gut, skin and urinary tract opportunistic pathogens. Pathogens: E. coli, Salmonella, Pseudomonas aeruginosa and S. aureus. Control represents growth of pathogen individually; treatment represents growth of pathogen in the presence of B. coagulans CGI314. **p<0.01, ***p<0.001, ****p<0.0001.

Fortispore has potential to control the presence of opportunistic pathogens in the gut and urinary tract where semi-liquid to liquid conditions will be common. Moreover, Fortispore has the potential to prevent the spread of opportunistic pathogens on dryer environments such as the human skin.

Fortispore has higher antimicrobial activity than the competitor B. coagulans probiotics The antimicrobial activity of B. coagulans CGI1314 (Fortispore) was compared with that of B. coagulans MTCC5856 (Lactospore®) and B. coagulans 6086 (BC30™) using the agar diffusion method against the following opportunistic and zoonotic pathogens of the human skin and the gut:
Escherichia coli 25922,
Salmonella enteritidis 13076,
Staphylococcus aureus RF122, and
Pseudomonas aeruginosa DSM3227.

B. coagulans CGI314 (Fortispore) demonstrated stronger antimicrobial activity against E. coli at 24 h and S. enteritidis at 48 h than BC30™ and Lactospore®. Fortispore may have a higher potential to control the presence of opportunistic and zoonotic pathogens that may be present in the gut or on the skin of humans: High antimicrobial activity of Fortispore against E. coli.

High antimicrobial activity of Fortispore against S. enteritidis at 48 hr.

No Antimicrobial activity detected across Bacillus coagulans against P. aeruginosa.

Limited antimicrobial activity of Bacillus coagulans against S. aureus using TSA agar.

Antimicrobial Activity—High Antimicrobial Activity Against E. coli:

B. coagulans CGI314 (Fortispore) had the strongest antimicrobial activity against E. coli tested using MRS agar. B. coagulans MTCC5856 (Lactospore®) and B. coagulans 6086 (BC30™) had limited antimicrobial activity.

TABLE 6
Larger zone of inhibition achieved with B. coagulans
CGI314 (Fortispore) strain against E. coli.

| | Zone of inhibition diameter (mm) against E. coli | | |
|---|---|---|---|
| | Rep 1 | Rep 2 | Rep 3 |
| Fortispore | 10 ± 0 | 9.5 ± 0.7 | 9 ± 1.4 |
| Lactospore ® | 2.5 ± 0.7 | — | 3 ± 0 |
| BC30 ™ | 4.5 ± 0.7 | 5 ± 1.4 | 4.5 ± 0.7 |

Values represent average inhibition±standard deviation (n=2).

FIG. 5 shows the strongest antimicrobial activity observed with B. coagulans CGI314 (Fortispore) against E. coli at 24 hr. (a) B. coagulans MTCC5856 (Lactospore®) (b) B. coagulans CG314 (Fortispore) (c) B. coagulans 6086 (BC30™) inoculated on MRS agar against E. coli 0.4% TSA agar overlay plates.

Fortispore inhibited the growth of the opportunistic intestinal and urinary tract pathogen E. coli.

Antimicrobial activity—High antimicrobial activity of against S. enteritidis at 48 hr:

At 24 h, B. coagulans 6086 (BC30™) had the strongest antimicrobial activity against S. enteritidis. However, at 48 h, B. coagulans CGI314 (Fortispore) had the strongest antimicrobial activity against this pathogen.

TABLE 7
Larger zone of inhibition achieved with B. coagulans CGI314
(Fortispore) strain against S. enteritidis at 48 hours.

| | | |
|---|---|---|
| Rep 1 | Rep 2 | Rep 3 |

Zone of inhibition diameter (mm) against S. enteritidis at 24 h

| | | | |
|---|---|---|---|
| Fortispore | 6.5 ± 2.1 | 4.5 ± 0.7 | 4.5 ± 0.7 |
| Lactospore ® | 3.5 ± 0.7 | — | — |
| BC30 ™ | 10 ± 0 | 9 ± 1.4 | 6 ± 1.4 |

Zone of inhibition diameter (mm) against S. enteritidis at 48 h

| | | | |
|---|---|---|---|
| Fortispore | 11 ± 1.4 | 13.5 ± 2.12 | 8 ± 1.4 |
| Lactospore ® | 4 ± 1.4 | — | — |
| BC30 ™ | 10 ± 0 | 9.5 ± 0.7 | 7 ± 1.41 |

Values represent average inhibition±standard deviation (n=2).

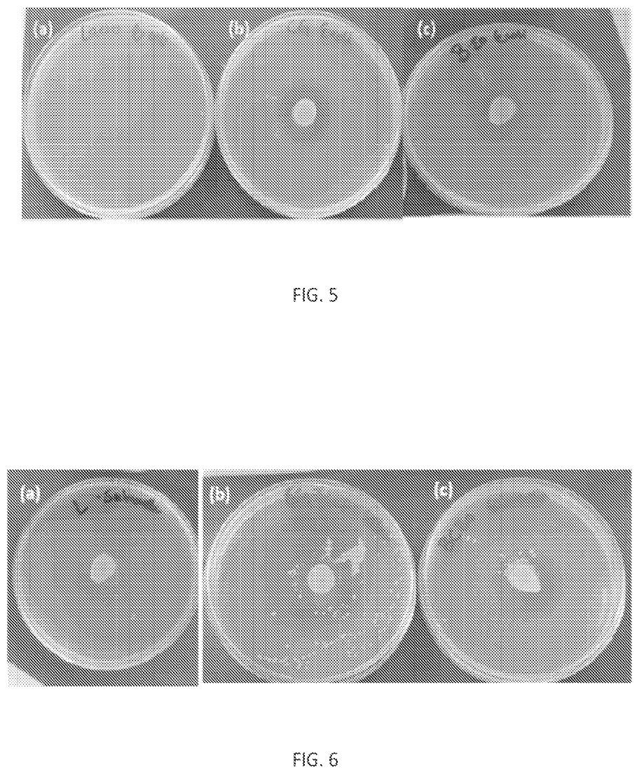

FIG. 6 shows the strongest antimicrobial activity observed with Fortispore against S. enteritidis at 48 hr. (a) Lactospore® (b) Fortispore (c) BC30™ inoculated on MRS agar against S. enteritidis 0.4% TSA agar overlay plates.

Fortispore inhibited the growth of the zoonotic pathogen Salmonella that is known to affect the intestinal tract.

Antimicrobial activity—no antimicrobial activity detected across Bacillus coagulans against P. aeruginosa:

Using the agar diffusion method, no antimicrobial activity was observed across B. coagulans against P. aeruginosa.

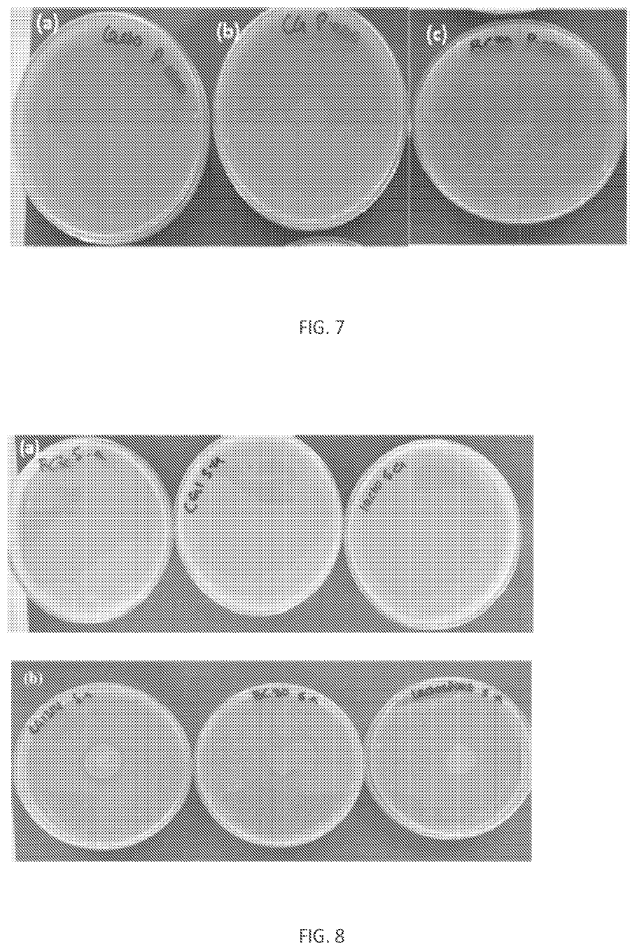

FIG. 7 shows no antimicrobial activity was detected across Bacillus coagulans against P. aeruginosa (a) B. coagulans MTCC5856 (Lactospore®) (b) B. coagulans CG314 (c) B. coagulans 6086 (BC30™) inoculated on TSA agar against P. aeruginosa 0.4% TSA agar overlay plates at 24 hr.

Fortispore does not release antimicrobials against skin pathogen P. aeruginosa. However, Fortispore shows potential activity against P. aeruginosa in liquid medium (see Table 5 and FIG. 7).

Antimicrobial activity—Limited antimicrobial activity of Bacillus coagulans against S. aureus using MRS Agar:

Using TSA agar, there was no evidence of antimicrobial activity by B. coagulans strains against the S. aureus. However, B. coagulans 6086 (BC30™) and B. coagulans CGI1314 (Fortispore) did demonstrate slight activity against this pathogen using MRS agar.

TABLE 8
Narrow zones of inhibitions were detected across the three B. coagulans
strains against S. aureus at 24 hours using MRS agar.

| | Zone of inhibition diameter (mm) against S. aureus | | |
|---|---|---|---|
| | Rep 1 | Rep 2 | Rep 3 |
| Fortispore | 3.5 ± 0.7 | 2.5 ± 0.7 | 4.5 ± 0.7 |
| Lactospore ® | — | — | — |
| BC30 ™ | 4 ± 1.4 | 3 ± 1.4 | 5.5 ± 2.1 |

Values represent average inhibition±standard deviation (n=2).

FIG. 8 shows limited antimicrobial detected across B. coagulans against S. aureus using MRS agar at 24 hr. (a) B. coagulans inoculated on TSA agar against S. aureus 0.4% TSA agar overlay plates. (Left to right: BC30, CGI314, Lactospore). (b) B. coagulans inoculated on MRS agar against S. aureus 0.4% TSA agar overlay plates. (Left to right: CGI314, BC30, Lactospore).

Fortispore can release limited amounts of antimicrobials on dry surfaces such as skin against skin pathogen S. aureus.

Fortispore is a potential antioxidant probiotic:

Total antioxidant activity of Fortispore B. coagulans was compared with L. rhamnosus. Fortispore has a higher antioxidant activity than L. rhamnosus.

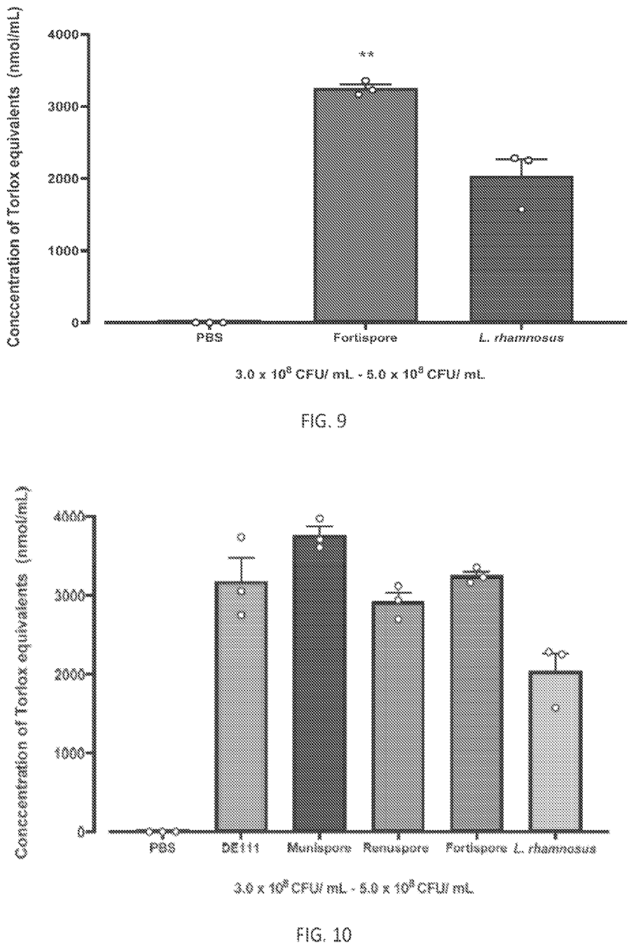

FIG. 9 shows total antioxidant capacity of B. coagulans and L. rhamnosus. Results show average concentration of Trolox equivalents in nmole/ml (n=3)±standard error.**p-value=0.0014.

The increased levels of Trolox equivalent concentration in Fortispore can neutralize and scavenge free radicals and prevent oxidative damage to the cells.

The Fortispore antioxidant activity is the second highest amount of antioxidant activity amongst all Deerland spores.

The total antioxidant activity of Fortispore B. coagulans was compared with B. megaterium MIT411, B. clausii CSI08, DE111 B. subtilis and L. rhamnosus. Fortispore has a high amount of antioxidant activity and is higher than L. rhamnosus.

FIG. 10 shows total antioxidant capacity of DE111, B. coagulans of the present invention, B. clausii CSI08, B. megaterium MIT411 and L. rhamnosus. Results show average concentration of Trolox equivalents in nmole/ml (n=3)±standard error.

The increased levels of Trolox equivalent concentration in Fortispore can neutralize and scavenge free radicals and prevent oxidative damage to the cells.

Fortispore (vegetative, non-spore form) did not adhere to intestinal epithelial cell line.

The adhesion ability of Bacillus coagulans strain (CGI314) to an in vitro model of intestinal epithelium was assessed. Adherence of B. coagulans strains CGI314 to HT-29 cell line was negligible at 37° C. Adhesion ability of B. coagulans CGI314 to the mucus producing cell line HT-29-MTX was negligible at 37° C.

FIG. 11 shows adherence of Fortispore to the HT-29 cell line was negligible.

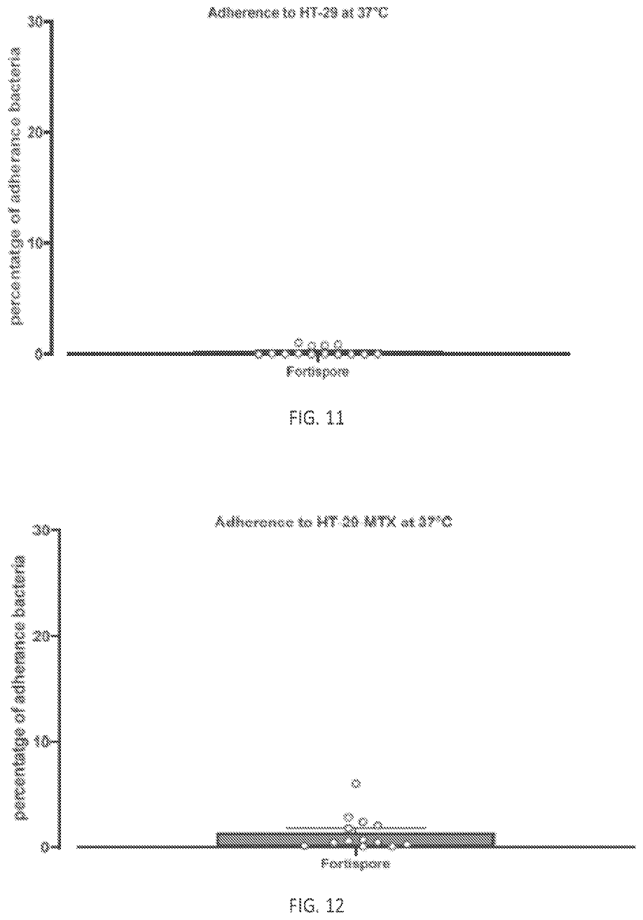

FIG. 12 shows adherence of Fortispore to the HT-29-MTX cell line is negligible.

Fortispore strain does not adhere to intestinal mucus or epithelial cells.

The adhesion ability of various Bacillus coagulans strains (CGI314, BC30™ Lactospore®) to an in vitro model of intestinal epithelium was assessed. Adherence of B. coagulans strains CGI314, BC30™, Lactospore® to HT-29 cell line is negligible at 37° C. Adhesion ability of B. coagulans BC30™ to the mucus producing cell line HT-29-MTX is higher than B. coagulans CGI314 and Lactospore® at 37° C.

FIG. 13 shows a study on the adherence of B. coagulans strains to the HT-29 cell line.

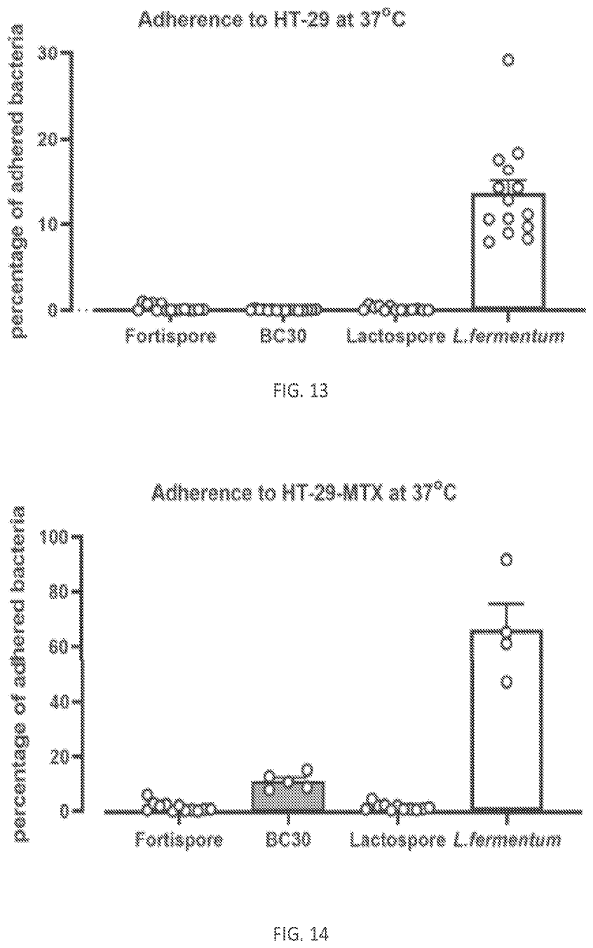

FIG. 14 show a study on the adherence of B. coagulans strains to the HT-29-MTX cell line.

B. coagulans strains (liquid culture, non-spore form) do not adhere to intestinal mucus or epithelial cells.

Fortispore displays low caseolytic activity

Fortispore was negative for caseolytic activity on Skim milk agar plates. Quantitative analysis of Fortispore caseolytic activity was evaluated by using a commercial kit employing fluorescently tagged casein derivatives. Fortispore displayed low extracellular protease activity.

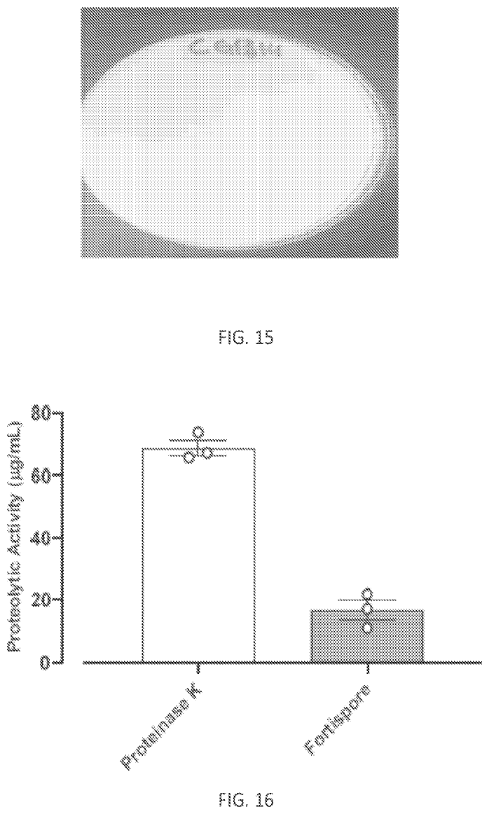

FIG. 15 shows a study on the absence of caseolytic activity in B. coagulans CGI1314 using both streak and overnight TSB broth method at 24 h or 48 h.

FIG. 16 shows proteolytic activity of Fortispore towards casein derivatives is lower than positive control Proteinase K using EnzCheck® kit following incubation at 37° C. for 24 h. Error bars present standard error (n=3).

Fortispore displays low protease activity towards Casein.

Amongst Deerland strains, Fortispore displays low extracellular protease activity towards casein derivatives.

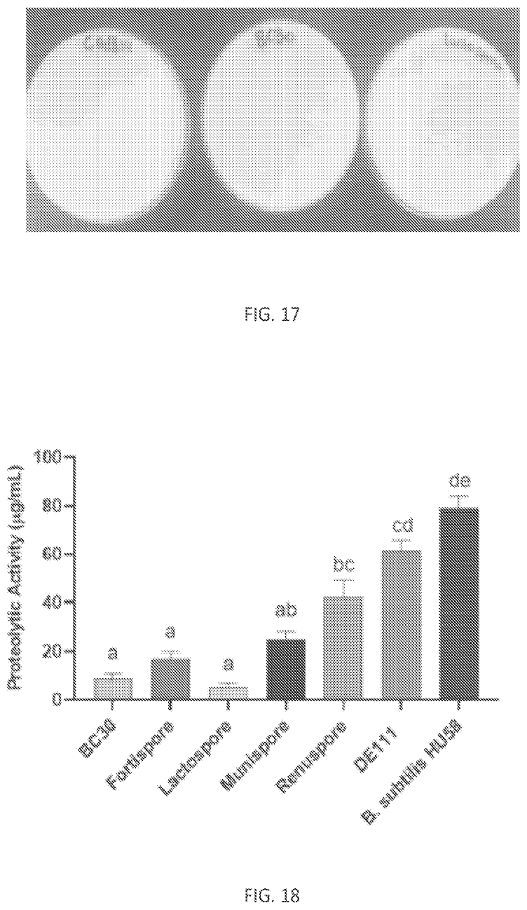

FIG. 17 shows absence of caseolytic activity across B. coagulans CGI1314, B. coagulans 6086 (BC30™) and B. coagulans MTCC5856 (Lactospore®) using both streak and overnight TSB broth method at 24 h or 48 h.

FIG. 18 shows Fortispore B. coagulans showing least protease activity towards casein amongst Deerland strains.

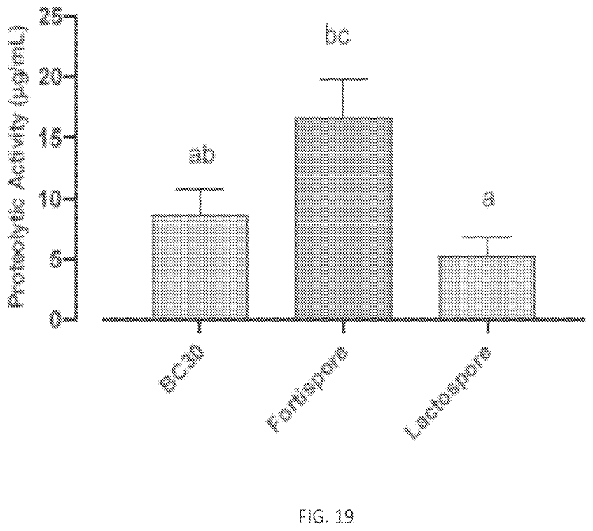

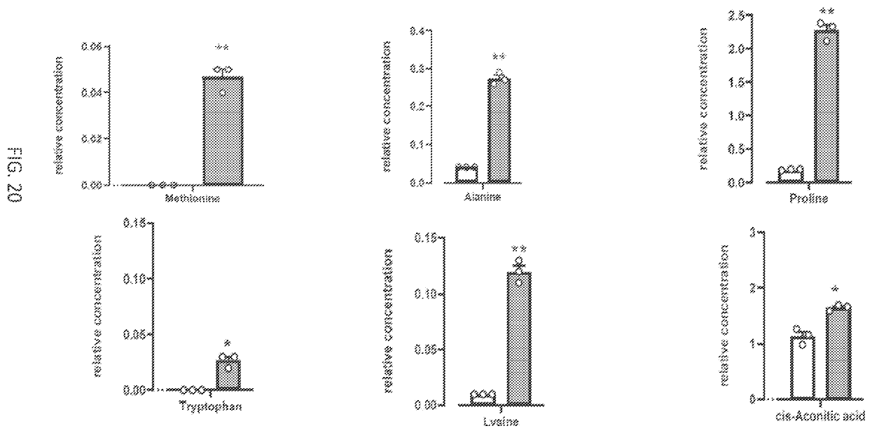

FIG. 19 shows a quantitative analysis of the caseolytic activity across B. coagulans strains determined by EnzCheck® kit following incubation at 37° C. for 24 h. Significance is indicated by a, b and/or c, multiple letters indicate results are not significantly different from more than one group (P<0.05). Error bars present standard deviations.

In comparison to the comparators (Lactospore® and BC30™), Fortispore displayed superior protease activity towards casein.

### Fortispore's Diverse Carbohydrate Profile

Fortispore can metabolize a range of monosaccharides, disaccharides, sugar alcohols, amine sugars and polysaccharides.

Fortispore was positive for metabolism of 22 carbohydrates out of 49 tested using commercial API 50 CH strip. The majority of these carbohydrates were simple sugars such as D-Ribose, D-Glucose, D-galactose, D-Fructose, D-Mannose, and Disaccharides such as Trehalose, Maltose and Cellobiose etc. Additionally, there were compounds belonging to glycosylated hydroquinone (arbutin), cyanogenic glycoside (amygladin) and alcoholic P3-glucoside (Salicin) that Fortispore can metabolise.

TABLE 9
List of Carbohydrates that are effectively fermented by B. Coagulans (Fortispore,
BC30 ™, Lactospore ®) using API 50 Ch strips

| | B. coagulans | | B. coagulans |
|---|---|---|---|
| Carbohydrate | CGI1314 | Carbohydrate | CGI1314 |
| 1-ARABINOSE | + | AMYGDALIN | + |
| D-RIBOSE | + | ARBUTIN | + |
| D-XYLOSE | + | ESCULIN FERRIC CITRATE | + |
| D-GALACTOSE | + | S ALICIN | + |
| D-GLUCOSE | + | D-CELLOBIOSE | + |
| D-FRUCTOSE | + | D-MALITOSE | + |
| D-MANNOSE | + | D-MELIBIOSE | − |
| L-RHAMNOSE | − | D-TREHALOSE | + |
| D-MANNITOL | + | AMIDON | + |
| D-S ORBITOL | + | GENTIOBIOSE | + |
| METHYL-AD- | + | D-ARABITOL | (+) |
| GLUCOPYRANOSIDE | | | |
| N-ACETYL | + | POTASSIUM GLUCONATE | + |
| GLUCOSAMINE | | | |

These data suggest that Fortispore could help digest these compounds in the gut.

Fortispore was strongly positive for D-sorbitol, and starch Amidon and the other two competitor's strains (B30™ and Lactospore®) were negative or weakly positive for these.

TABLE 10
List of carbohydrates that are effectively fermented by B. Coagulans (Fortispore, BC30 ™, Lactospore ®) using API 50 Ch strips

| | B. | B. | | | B. | B. | |
|---|---|---|---|---|---|---|---|
| | coagulans | coagulans | B. | | coagulans | coagulans | B. |
| | 6086 | MTCC5856 | coagulans | | 6086 | MTCC5856 | coagulans |
| Carbohydrate | (BC30TM) | (Lactospore ®) | CGI1314 | Carbohydrate | (BC30TM) | (Lactospore ®) | CGI1314 |
| 1-ARABINOSE | + | + | + | AMYGDALIN | + | + | + |
| D-RIBOSE | + | + | + | ARBUTIN | + | + | + |
| D-XYLOSE | + | + | + | ESCULIN FERRIC CITRATE | + | + | + |
| D-GALACTOSE | + | + | + | SALICIN | + | + | + |
| D-GLUCOSE | + | + | + | D-CELLOBIOSE | + | + | + |
| D-FRUCTOSE | + | + | + | D-MALTOSE | + | + | + |
| D-MANNOSE | + | + | + | D-MELIBIOSE | + | + | − |
| L-RHAMNOSE | + | + | − | D-TREHALOSE | + | + | + |
| D-MANNITOL | − | + | + | AMIDON | (+) | − | + |
| D-SORBITOL | (+) | − | + | GENTIOBIOSE | + | + | + |
| METHYL-AD- | + | + | + | D-ARABITOL | − | (+) | (+) |
| GLUCOPYRANOSIDE | | | | | | | |
| N-ACETYLGLUCOSAMINE | + | + | + | POTTASIUM GLUCONATE | + | + | + |

These data suggest that Fortispore could help digest these compounds in the gut.

Fortispore has esterase, peptidase, phosphatase and glucosidase activity:

Fortispore was positive for esterase, peptidase, phosphatase, and galactosidase activity using API ZYM kit which implies:
a high possibility of Fortispore to generate free fatty acids from the action of esterase in the presence of an appropriate lipid source.
Galctosidase adds to the carbohydrate catabolism potential of Fortispore as these are active against various oligosaccharides, lactosylceramides, lactose, and numerous glycoproteins.
Aminopeptidases which catalyze the hydrolysis of leucine, valine and cysteine substrates.

Indeed, in silico analysis have identified genes encoding esterases, peptidases, and galactosidases.

TABLE 11
Enzymatic profile of Fortispore using API ZYM kit

| | | B. coagulans |
|---|---|---|
| | | CGI1314 (Fortispore) |
| Esterase activity | Esterase (C4:0) | + |
| | Esterase (C8:0) | + |
| Lipase activity | Lipase (C14:0) | − |
| Peptidase activity | Leucine arylamidase | + |
| | Valine arylamidase | + |
| | Cystine arylamidase | + |
| Proteinase activity | Trypsin | − |
| | α-chymotrypsin | − |
| Phosphatase activity | Acid phosphatase | + |
| | Alkaline phosphatase | + |
| | Phosphohydrolyase | + |
| Glycosidase activity | α-Galactosidase | + |
| | β-Galactosidase | + |
| | β-Glucuronidase | − |
| | α-Glucosidase | + |
| | β-Glucosidase | + |
| | β-Glucosaminidase | − |
| | α-Mannosidase | − |
| | α-Fucosidase | − |

This study confirms the hydrolytic abilities of Fortispore towards oligosaccharides and indicates the potential to break down fats and peptides to release free amino acids.

These data suggest that Fortispore could help digest these molecules in the gut.

B. coagulans enzymatic profile indicates peptidase and esterolytic capability and confirms oligosaccharide degradation capability:

All B. coagulans were positive for esterase, peptidase, phosphatase, and galactosidase activity using API ZYM kit which implies:
a high possibility of B. coagulans to generate free fatty acids from the action of esterase in the presence of an appropriate lipid source.
Galactosidase adds to the carbohydrate catabolism potential of B. coagulans as these are active against various oligosaccharides, lactosylceramides, lactose, and numerous glycoproteins.
Aminopeptidases which catalyze the hydrolysis of leucine, valine and cysteine substrates.

TABLE 12
Enzymatic profile of Fortispore using API ZYM kit

| | | B. coagulans | B. coagulans | B. coagulans |
|---|---|---|---|---|
| | | CGI1314 | MTCC5856 | 6086 |
| | | (Fortispore) | (Lactospore ®) | (BC30 ™) |
| Esterase activity | Esterase (C4:0) | + | + | + |
| | Esterase (C8:0) | + | + | + |
| Lipase activity | Lipase (C14:0) | − | − | − |
| Peptidase activity | Leucine arylamidase | + | + | + |
| | Valine arylamidase | + | + | + |
| | Cystine arylamidase | + | + | + |
| Proteinase activity | Trypsin | − | − | − |
| | α-chymotrypsin | − | − | − |
| Phosphatase activity | Acid phosphatase | + | + | + |
| | Alkaline phosphatase | + | + | + |
| | Phosphohydrolyase | + | + | + |
| Glycosidase activity | α-Galactosidase | + | + | + |
| | β-Galactosidase | + | + | + |
| | β-Glucuronidase | − | − | − |
| | α-Glucosidase | + | + | + |
| | β-Glucosidase | + | + | + |
| | β-Glucosaminidase | − | − | − |
| | α-Mannosidase | − | − | − |
| | α-Fucosidase | − | − | − |

This study demonstrates no differences in hydrolytic abilities of B. coagulans strains towards several enzymes using a commercial API ZYM Kits.

Fortispore releases free amino acids from glycolysis and protease metabolism

Metabolomic analysis reveals Fortispore fermentation and proteolytic capability towards milk carbohydrates and proteins generating a range of amino acids.

UHT Milk model was used to analyse the carbohydrate fermentation and proteolytic abilities of Fortispore. GC-MS analysis identified a total of 38 free amino acids (FAA) compounds, of which 10 were found to be statistically significant in Fortispore. A few of the carboxylic acids identified are associated with carbohydrate metabolism pathway, which confirms the presence of a Lactose/galactose/glucose uptake and enzymatic metabolism system to be active in Fortispore. Additionally, there is also evidence of an active proteolytic system in Fortispore as there is a release of a few amino acid linked with peptidase activity and benzoic acid which is linked with further catabolism of phenylalanine.

TABLE 13
Statistically significant compounds analysed with GC-MS using
the FAA method are listed along with potential precursors

| Compound | Potential peptidases/precursors |
|---|---|
| Alanine | Aminopeptidases PepN |
| Proline | Pep P/PepX |
| Methionine | MAP/PepM |
| Lysine | Aminopeptidases PepN |
| Tryptophan | Aminopeptidases |
| Succinic acid | Lactose/glucose |
| Lactic Acid | Lactose/glucose |
| Cis-Aconitic acid | Lactose/glucose |
| Isocitric acid | Lactose/glucose |

Overall, the enzymatic and metabolomic data suggest that Fortispore has active carbohydrate-metabolizing enzymes, as most of the compounds generated are those from the glycolysis pathway (conversion of carbohydrates in milk). Additionally, there is evidence of potentially active aminopeptidases, lyases and decarboxylases generating carboxylic acids and releasing amino acids from Fortispore fermented UHT milk.

Fortispore releases free amino acids from glycolysis and protease metabolism

### Free Amino Acid Analysis Presented in Bar Graphs (Mean+SEM)

FIG. 20 shows FAA profile of Fortispore UTH fermented milk. P-value≤0.005=** and P-value≤0.01=*. The white bar represents the control.

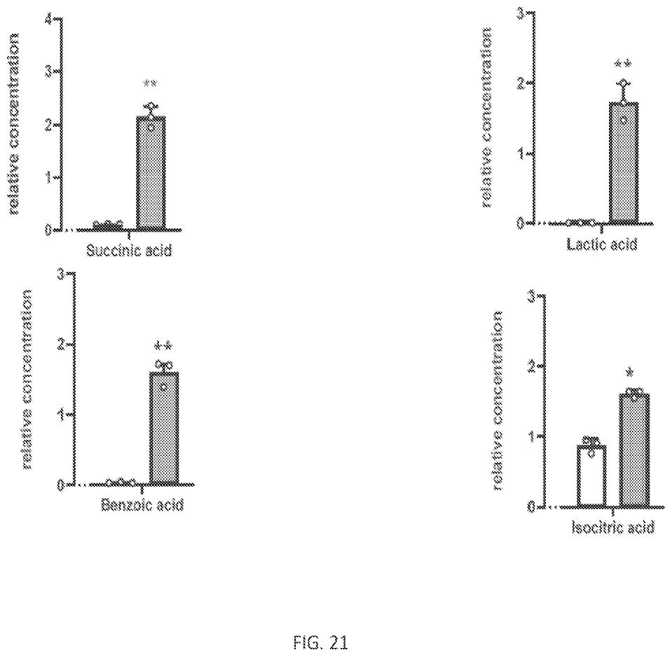

FIG. 21 shows FAA profile of Fortispore UTH fermented milk. P-value≤0.005=** and P-value≤0.01=*. The white bar represents the control.

Fortispore is slightly more efficient in producing compounds beneficial in skin health in comparison to competitor B. coagulans strains

Ganeden Biotech markets B. coagulans 6086 (BC30™) as an anti-aging probiotic,as it produces maximum amounts of naturally derived L+ lactic acid, bacteriocins, hydrogen peroxide, enzymes and other metabolites. Lactic acid is associated with anti-aging, fighting acne due to its antimicrobial activity and hydration. Succinic acid is also associated with anti-inflammatory, antimicrobial and hydration. Benzoic acid is associated with anti-microbial activity.

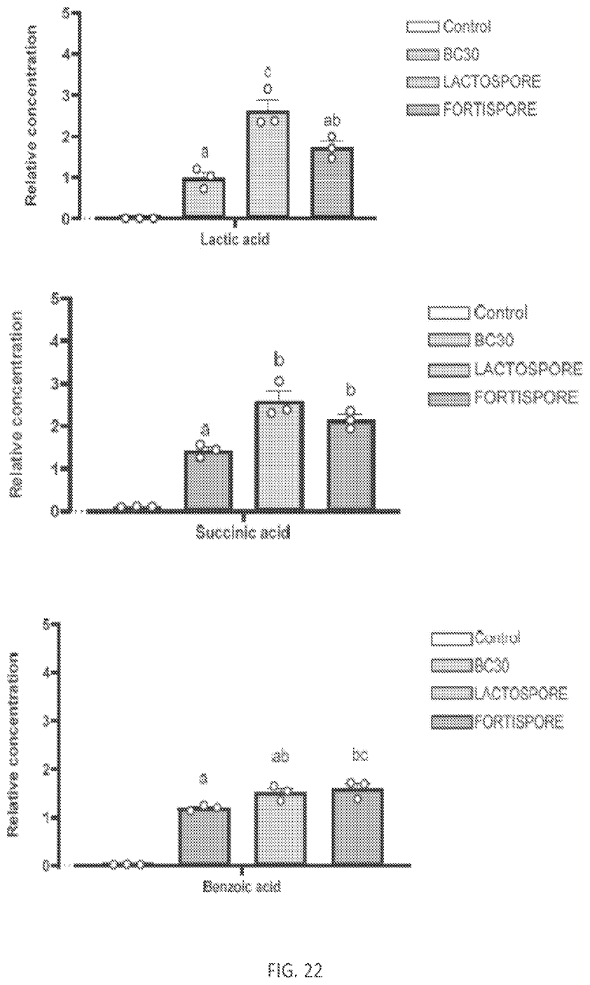

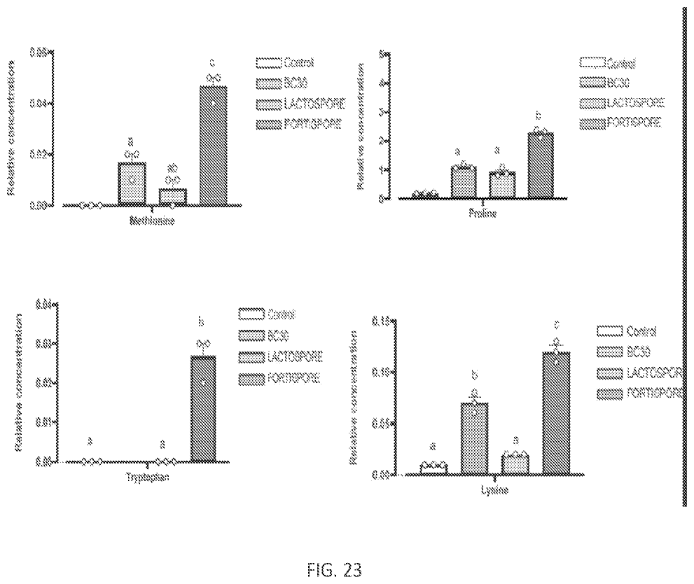

FIG. 22 compares Fortispore with Lactospore and BC30 in the production of lactic acid, succinic acid and Benzoic acid. Significance is indicated by a, b and/or c, multiple letters indicate results are not significantly different from more than one group (PC 0.05). Error bars present standard deviations.

Our results show Fortispore to be slightly more efficient than BC30™ in terms of lactic acid production and superior in succinic acid and benzoic acid production.

Fortispore can produce amino acids beneficial in skin health in comparison to competitor B. coagulans strains

Fortispore is compared with Lactospore® and BC30. Our results show Fortispore to be superior to BC30™ and Lactospore® in producing methionine, proline, lysine and tryptophan.

Methionine is a sulfur-containing amino acid that improves the tone and elasticity of the skin, promotes healthy hair and strengthens the nails. Proline helps lower inflammation, which promotes a healthy immune system. It also helps trigger a cascade of anti-inflammatory compounds and genes that help with recovery. Lysine is an essential amino acid with many benefits that range from preventing cold sores to reducing anxiety and promoting wound healing. Tryptophan is associated with relief from depression and anxiety.

FIG. 23 compares Fortispore with Lactospore and BC30 in the production of amino acids. Significance is indicated by a, b and/or c, multiple letters indicate results are not significantly different from more than one group (P<0.05). Error bars present standard deviations.

Fortispore proteomics analysis identifies proteins with potential probiotic benefits

Extracellular secretions of Fortispore were sent to mass spectrometry to identify proteins released by the probiotic strain. A total of 28 proteins were detected of which 6 had potential probiotic benefits.

TABLE 14
Fortispore proteomics identifies proteins
with potential probiotic benefits

| Protein of interest | Potential role |
|---|---|
| Hydrolase Nlp/P60 | Involved in degradation of proteins or fats |
| Serine protease | Involved in digestion of proteins - cleaves |
| | peptide bonds |
| Peptidase M23 | Involved in digestion of proteins |
| | Antimicrobial properties - lysis of bacterial |
| | cell wall |
| Chaperonin GroEL | Involved in protein folding |
| Peptidase | Involved in digestion of proteins |
| Dextransucrase | Involved in breakdown of starch and sucrose |

These data confirm previous in vitro results showing how Fortispore can help in digestion of proteins and carbohydrates and has antimicrobial properties against pathogens.

Fortispore growth increased in the presence of Fibersol® in minimal media.

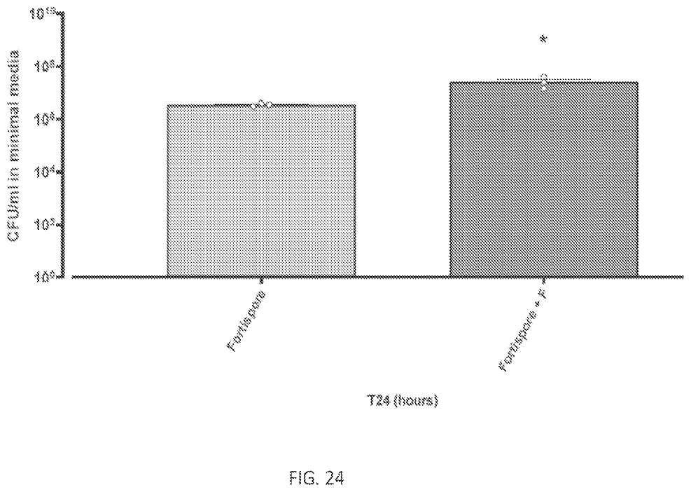

FIG. 24 shows Fibersol® significantly increased the concentration (CFU/mL) of Fortispore by 1 log₁₀ in minimal media 24 hours post incubation compared to controls. *p<0.05

There is a significant increase in the growth of Fortispore in the presence of Fibersol® in minimal media in comparison to competitor B. coagulan strains.

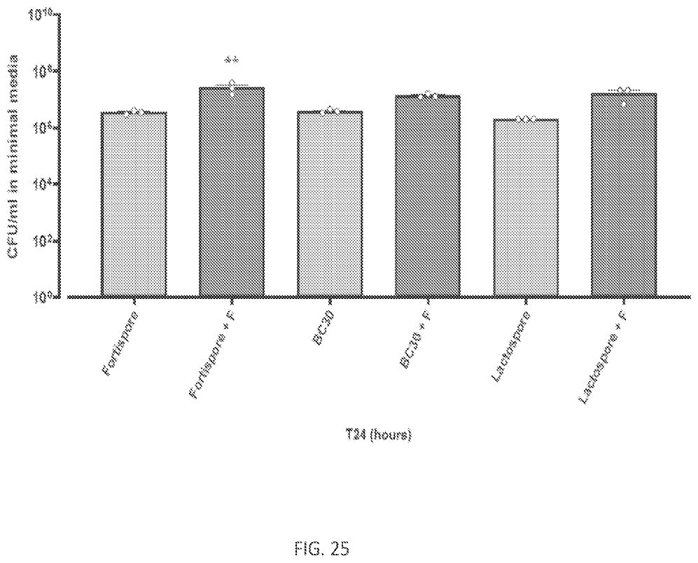

FIG. 25 shows Fibersol® significantly increases the concentration (CFU/mL) of Fortispore in Minimal media whereas no significance in the growth of BC30 and Lactospore were seen. Statistical analysis conducted using one-way ANOVA using Tukey method. **p-value=<0.01.

There was no increase in the growth of Fortispore in the presence of Fibersol® in TSB media.

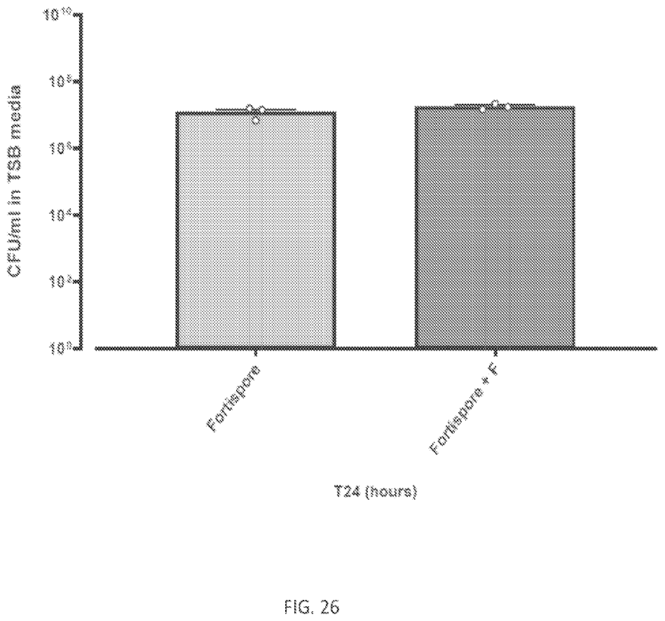

FIG. 26 shows that Fibersol® did not show significant increase in the concentration (CFU/mL) of Fortispore in TSB media compared to controls. Unlike minimal media, Fibersol® didn't increase the growth of Fortispore in rich media, likely because they reach maximum growth with the nutrients present in TSB media.

The growth of Fortispore, BC 30, DE111 and pathogens were compared in the presence of Fibersol® in TSB media.

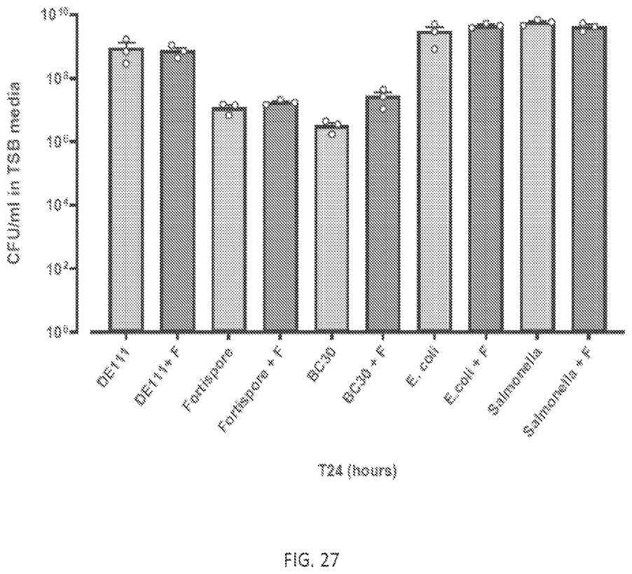

FIG. 27 shows Fibersol® did not show significant increase in the concentration (CFU/mL) of DE111, Fortispore, BC30, E. coli and Salmonella enteritidis in TSB media compared to controls. Unlike minimal media, Fibersol® didn't increase the growth of DE111, Fortispore, BC30, E. coli and S. enteritidis in rich media, most likely because they reach maximum growth with the nutrients present in TSB media.

There was no increase in the growth of Fortispore in the presence of Fibersol® in 50% TSB media.

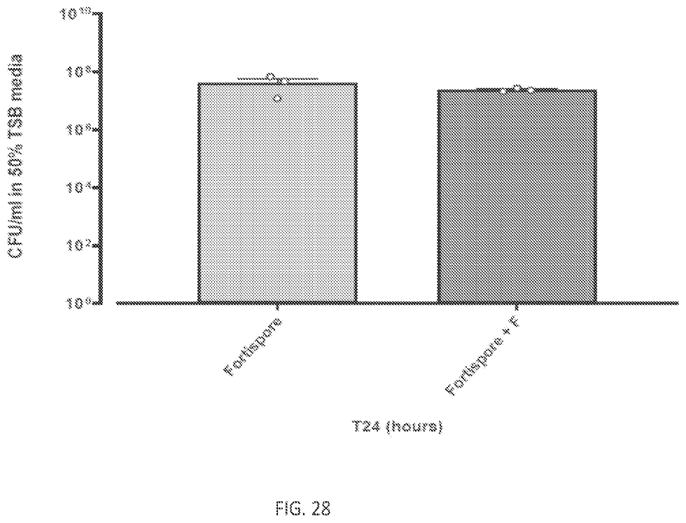

FIG. 28 shows Fibersol® did not significantly increase the concentration (CFU/mL) of Fortispore in 50% TSB media compared to controls.

The growth of Fortispore, BC 30 and DE111 was compared in the presence of Fibersol® in 50% TSB media.

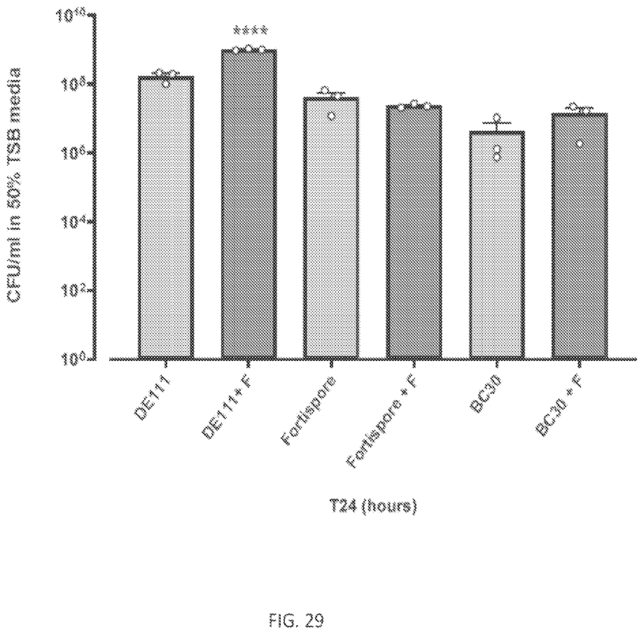

FIG. 29 shows Fibersol® significantly increased the yield (CFU/mL) of DE111 by 1 log₁₀ after 24 hours in 50% TSB media. ****p<0.0001. Fibersol® did not show significant increase in the concentration (CFU/mL) of Fortispore and BC30 in 50% TSB media compared to controls.

No significant growth of Fortispore was observed in the presence of Fibersol® supplemented BHI media.

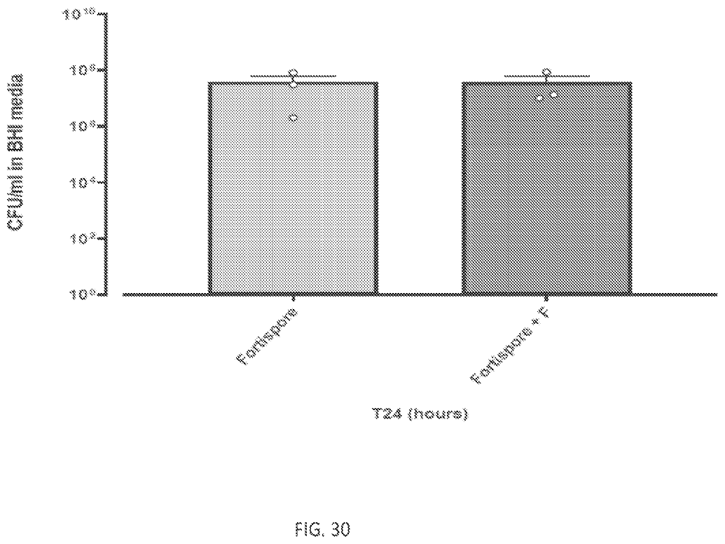

FIG. 30 shows that Fibersol® did not show significant increase in the concentration (CFU/mL) of Fortispore compared to controls in BHI media. Unlike minimal media, Fibersol® didn't increase the growth of Fortispore in rich media, most likely because they reach maximum growth with the nutrients present in BHI media.

The growth of Fortispore, BC 30 and DEl11 was compared in the presence of Fibersol® in BHI media.

FIG. 31 shows that Fibersol® did not show significant increase in the concentration (CFU/mL) of DE111, Fortispore and BC30 compared to controls in BHI media. Unlike minimal media, Fibersol® didn't increase the growth of DE111, Fortispore and BC30 in rich media, most likely because they reach maximum growth with the nutrients present in BHI media.

No increase in the growth of Fortispore was observed in the presence of Fibersol® in 50% BHI media.

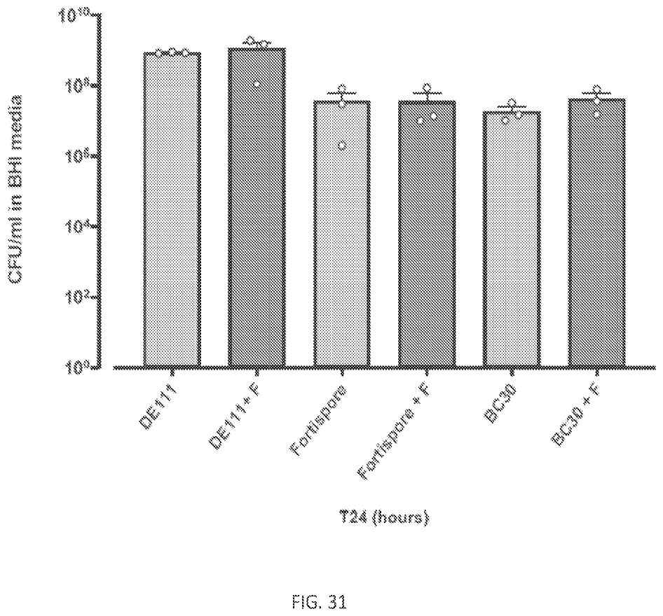

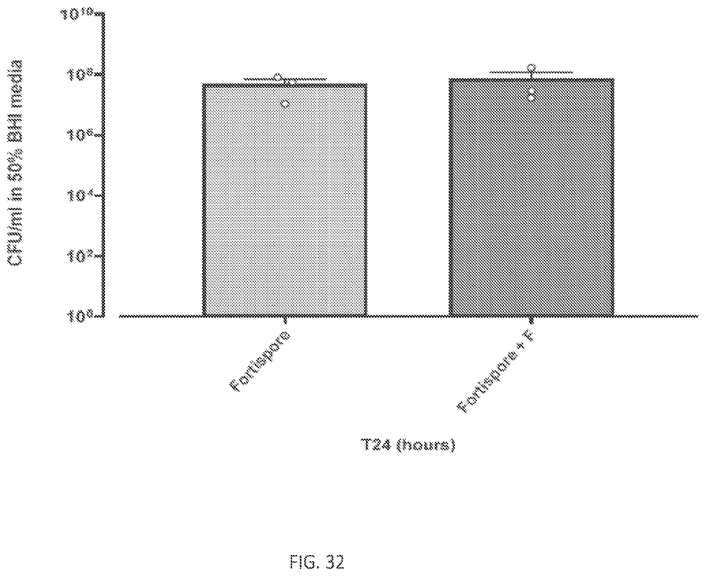

FIG. 32 shows that Fibersol® did not show significant increase in the concentration (CFU/mL) of Fortispore compared to controls in 50% BHI media. Unlike minimal media, Fibersol® didn't increase the growth of Fortispore in rich media, most likely because they reach maximum growth with the nutrients present in BHI media.

No increase in the growth of Fortispore, BC 30 and DEl11 was observed in the presence of Fibersol® in 50% BHI media.

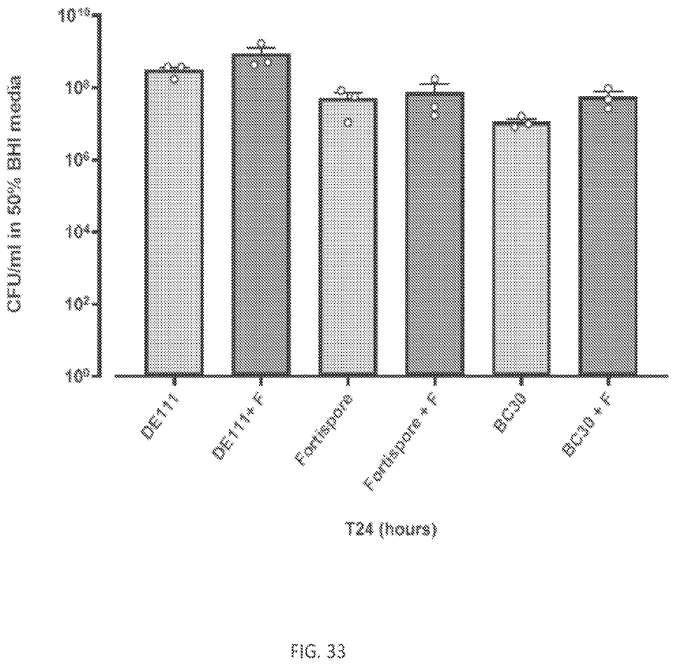

FIG. 33 shows that Fibersol® did not show significant increase in the concentration (CFU/mL) of DE111, Fortispore and BC30 compared to controls in 50% BHI media. Unlike minimal media, Fibersol® didn't increase the growth of DE111, Fortispore and BC30 in rich media, most likely because they reach maximum growth with the nutrients present in BHI media.

### Example 2

### Assess Adhesion Ability to an In Vitro Model of Intestinal Epithelium

Cell lines: Human Colorectal Adenocarcinoma Cell Line HT-29 and mucous-secreting cell line HT-29-MTX were propagated using low glucose DMEM medium supplemented with 10% Fetal Bovine Serum, 2 mM glutamine, 100 U/ml penicillin, 100 μg/l streptomycin, and 2 μg/ml amphotericin B in a 5% CO₂ atmosphere at 37° C.

Cells were seeded onto 24-well plates at a density 5×10⁵ cell/well and cultured for 21-28 days to complete maturation. Media was replaced every 2-3 days.

Prior to experiments cells were washed twice with 0.5 ml DPBS. DPBS was completely aspirated from the wells after the second round of washing.

Preparation of spores: Ten milligrams of B. clausii CSI08, B. megaterium MIT411 and B. coagulans CGI314 spores powders were weighted in 15 ml falcon tubes and resuspended in 10 ml of full culture medium without antibiotics. Suspensions were aliquoted and stored at −20° C. until use. Suspensions were used within 2 weeks upon preparation.

Adhesion assay: 500 μl of spores suspensions (1.3×10⁷-9.2×10⁷ CFU/ml) were added to HT-29 and HT-29-MTX cells, mixed by a gentle swirl, and incubated for 2.5 h at 37° C. in the CO₂ incubator. Control wells not containing mammalian cells were prepared and incubated in parallel in the same way (0.5 ml of spores' suspensions).

Upon incubation HT-29 and HT-29-MTX cells were washed 4 times with 0.5 ml PBS. After that 50 μl of Trypsin/EDTA solution and 50 μl of PBS were added to the wells and incubated for 10 min with gentle shaking (˜100 rpm) at 37° C. Fifty microliters of Trypsin/EDTA solution were added to control wells.

Consequently, 450 μl of PBS were added to the wells with spores, contents of the wells were transferred into Eppendorf tubes with scrapping and subjected to three rounds of vigorous shaking 30 sec each. Contents of control wells were transferred into Eppendorf tubes and subjected to one round of shaking.

Serial dilutions (plus dilutions of control wells) were prepared in PBS and plated onto BC agar (B. coagulans CGI314) or PetriFilm™ (B. clausii CSI08, B. megaterium MIT411). Plates were incubated at 37° C. for 48 h prior to counting, PetriFilm were incubated at 37° C. for 24 h prior to counting.

Experiments were performed two or three times with three technical replicates per experiment. The results are expressed as means±SEM.

TABLE 15
Adherence of B. clausii CSI08, B. megaterium MIT411 and
B. coagulans CGI314 spores to the HT-29-MTX cell line

| Percentage of | | | |
|---|---|---|---|
| adherence to HT-29- | B. clausii | B. megaterium | B. coagulans |
| MTX cell line | CSI08 | MIT411 | CGI314 |
| Mean | 12.10 | 20.68 | 19.68 |
| Standard error | 0.7710 | 3.005 | 2.197 |
| of the mean | | | |

TABLE 16
Adherence of B. clausii CSI08, B. megaterium MIT411
and B. coagulans CGI314 spores to the HT-29 cell line

| Percentage of | | | |
|---|---|---|---|
| adherence to HT-29 | B. clausii | B. megaterium | B. coagulans |
| cell line | CSI08 | MIT411 | CGI314 |
| Mean | 0.2578 | 1.499 | 0.8033 |
| Standard error | 0.02035 | 0.2983 | 0.1781 |
| of the mean | | | |

### Conclusion

1. Results set out above demonstrate higher ability of spores to adhere to the mucous-secreting cell line HT-29-MTX compared to non-mucus secreting cells, possibly due to spores' physical properties.
2. B. megaterium MIT411 and B. coagulans CGI314 spores have higher (but overall low) ability to adhere to non-mucus producing cell line HT-29 compared to B. clausii CSI08 spores.

### Example 3

### Evaluation of Bacillus coagulans CGI314 on Safety, Tolerance and Gastrointestinal Health: A Randomised, Double-Blind, Placebo-Controlled Trial in Healthy Adults

### Study Interventions

Probiotic 1 containing 1×10⁹ CFU of Bacillus coagulans CGI314 and Bacillus subtilis DE111® administered daily,
Probiotic combination containing about (and greater than) 0.5×10⁹ CFU of Bacillus subtilis DEl111, about 0.5×10⁹ CFU of Bacillus megaterium MIT411, about (and less than) 0.5×10⁹ CFU Bacillus coagulans CGI314, about 0.5×10⁹ CFU Bacillus clausii
CSI08; 2×10⁹ Bacillus spores total administered daily
Placebo: rice maltodextrin administered daily

### Indication Studied

Gastrointestinal homeostasis and immune system.

### Study Description

Healthy adults aged 18-65 years were recruited and screened for participation in the study. Eligible participants were randomized 1:1:1: to either one of the two experimental groups or control group and underwent 45 days of treatment and 2 weeks of follow-up to evaluate safety and efficacy of new probiotic strains in healthy adults.

For the purpose of this investigational products were packed in individual capsules, that corresponded to the daily dose. Each capsule contained 300 mg of ingredients:
Probiotic formula 1: Bacillus coagulans CGI314 and Bacillus subtilis DE11® (1×10⁹ CFU total), ˜4.17 mg; Low Moisture Rice Maltodextrin, 292.23 mg; Medium Chain Triglycerides, 3.6 mg.
Probiotic cocktail: 2×10⁹ CFU Bacillus spores, including Bacillus subtilis DE111® (about (and greater than) 0.5×10⁹ CFU), 2.1 mg; Bacillus megaterium MIT411 (about 0.5×10⁹ CFU), 2.1 mg; Bacillus clausii CSI08 (about 0.5×10⁹ CFU), 2.1 mg; Bacillus coagulans CGI314 (about (and less than) 0.5×10⁹ CFU), 2.1 mg; Low Moisture Rice Maltodextrin, 288 mg; Medium Chain Triglycerides, 3.6 mg.
Placebo consisted of all the same ingredients as investigational product only without probiotic organisms. Placebo was in individual capsules, that corresponded to the daily dose. Each capsule contained 300 mg of ingredients: Placebo: Low Moisture Rice Maltodextrin, 296.4 mg; Medium Chain Triglycerides, 3.6 mg.

Table 17 summarizes the change in scores of the Gut-brain axis questionnaire from baseline to the end of the treatment period, for the 4 probiotic groups and the placebo group. Mean changes with 95% confidence interval are shown. Results of the ANOVA omnibus test (p*-value) and one-sample T test (p-value) are also presented. Test of normality for the change in scores of the Gut-brain axis show that the data do not follow normal distribution, which could affect the results with borderline significance (p-values between 0.05 and 0.10). This affects two items: Loss of energy and Changes in appetite. An alternative nonparametric Kruskal Wallis test was applied to these items; p-values of 0.111 (Loss of energy) and 0.123 (Changes in appetite) were observed.

In general, mean values of the score changes of the Gut-brain axis questionnaire are negative for all the symptoms tested, meaning that the symptoms were less intense (participants were less bothered by these symptoms) at the end of the treatment period. One-sample T-test results show that in one third of tests (of 70 performed) a statistically significant change Gut-brain axis questionnaire score was observed. However, this can be observed for all treatment groups including the placebo group. Consequently, the results of the ANOVA test show, that no significant differences in Gut-brain axis score change among the treatment groups were detected, however a borderline significance for the items Loss of energy and Changes in appetite was observed. The participants in the Bacillus megaterium group experienced the largest change for these two items.

TABLE 17
Change in scores of the Gut-brain axis questionnaire items from
baseline to the end of the treatment period: Pairwise comparison
of probiotic groups with placebo (post hoc ANOVA: Dunnet t-test)

| | Bacillus | Probiotic |
|---|---|---|
| | coagulans | cocktail |
| | (N* = 25) | (N* = 25) |
| Sadnesschange | | |
| mean difference vs placebo | −0.42 (−1.00; | −0.44 (−1.02; |
| (95% CI) | 0.17) | 0.14) |
| p-value vs placebo | 0.236 | 0.192 |
| Irritationchange | | |
| Mean difference vs placebo | −0.33 (−0.94; | −0.35 (−0.95; |
| (95% CI) | 0.27) | 0.25) |
| p-value vs placebo | 0.453 | 0.405 |
| Loss of energychange | | |
| Mean difference vs placebo | 0.08 (−0.57; | −0.02 (−0.67; |
| (95% CI) | 0.74) | 0.63) |
| p-value vs placebo | 0.993 | 1.000 |
| Changes in apetitechange^{a} | | |
| Mean difference vs placebo | −0.33 (−1.01; | −0.04 (−0.54; |
| (95% CI) | 0.34) | 0.46) |
| p-value vs placebo | 0.390 | 0.999 |
| Hard to | | |
| breathe/chokingchange | | |
| Mean difference vs placebo | −0.21 (−0.61; | −0.16 (−0.56; |
| (95% CI) | 0.19) | 0.24) |
| p-value vs placebo | 0.505 | 0.709 |
| Heart | | |
| pounding/racingchange^{a} | | |
| Mean difference vs placebo | −0.33 (−0.92; | 0.08 (−0.35; |
| (95% CI) | 0.25) | 0.51) |
| p-value vs placebo | 0.176 | 0.968 |
| Sleeping problemschange | | |
| Mean difference vs placebo | −0.04 (−0.66; | −0.15 (−0.76; |
| (95% CI) | 0.57) | 0.46) |
| p-value vs placebo | 0.999 | 0.926 |
| Concentration | | |
| problemschange | | |
| Mean difference vs placebo | 0.13 (−0.42; | −0.02 (−0.56; |
| (95% CI) | 0.67) | 0.51) |
| p-value vs placebo | 0.944 | 1.000 |
| Nervousness/stresschange | | |
| Mean difference vs placebo | 0.00 (−0.71; | −0.14 (−0.85; |
| (95% CI) | 0.71) | 0.56) |
| p-value vs placebo | 1.000 | 0.963 |
| Angriness/tensionchange | | |
| Mean difference vs placebo | −0.04 (−0.63; | −0.35 (−0.93; |
| (95% CI) | 0.54) | 0.23) |
| p-value vs placebo | 0.999 | 0.372 |
| Headachechange | | |
| Mean difference vs placebo | −0.17 (−0.76; | −0.03 (−0.61; |
| (95% CI) | 0.42) | 0.55) |
| p-value vs placebo | 0.892 | 1.000 |
| Muscle aches/painchange | | |
| Mean difference vs placebo | 0.33 (−0.17; | 0.13 (−0.37; |
| (95% CI) | 0.84) | 0.63) |
| p-value vs placebo | 0.293 | 0.912 |
| Stiffnesschange | | |
| Mean difference vs placebo | 0.08 (−0.33; | 0.17 (−0.24; |
| (95% CI) | 0.50) | 0.58) |
| p-value vs placebo | 0.965 | 0.692 |
| Dizziness change | | |
| Mean difference vs placebo | −0.13 (−0.49; | 0.00 (−0.36; |
| (95% CI) | 0.24) | 0.36) |
| p-value vs placebo | 0.807 | 1.000 |

N* = number of participants included in the ITT population,
*p-value for Dunnet T-test (pairwise comparison)

Mean differences with 95% confidence intervals and p-values of pairwise comparison between individual probiotic group and placebo group are shown in Table 17. Compared to placebo, none of the study products containing probiotics showed a statistically significant difference in Gut-brain axis score change from baseline to the end of treatment period.

Mean differences with 95% confidence intervals and p-values of pairwise comparison between individual probiotic group and placebo group are shown in Table 18. Compared to placebo, none of the study products containing probiotics showed a statistically significant difference in the sum of Gut-brain axis items scores at baseline or at the end of treatment period.

TABLE 18
The sum of scores of the Gut-brain axis questionnaire items at baseline
and at the end of the treatment period: Pairwise comparison of probiotic
groups with placebo (post hoc ANOVA: Dunnet t-test)

| | Bacillus | Probiotic |
|---|---|---|
| | coagulans | cocktail |
| | (N* = 25) | (N* = 25) |
| Sum of Gut brain axis items | | |
| scores at baseline | | |
| Mean difference vs placebo | 0.43 (−3.86; | −0.02 (−4.30; |
| (95% CI) | 4.71) | 4.27) |
| p-value vs placebo | 0.997 | 1.000 |
| Sum of Gut-brain axis items | | |
| scores at the end of | | |
| the treatment period | | |
| Mean difference vs placebo | −1.00 (−4.62; | −1.34 (−4.92; |
| (95% CI) | 2.62) | 2.24) |
| p-value vs placebo | 0.899 | 0.762 |

N* = number of participants included in the ITT population,
*p-value for Dunnet T-test (pairwise comparison)

Mean differences with 95% confidence intervals and p-values of pairwise comparison between individual probiotic group and placebo group are shown in Table 19. Compared to placebo, none of the study products containing probiotics showed a statistically significant difference in HDL, LDL, TC or TG change from baseline to the end of treatment period.

TABLE 19
Cholesterol and triglyceride change from baseline to the end of
the treatment period in ITT population: Pairwise comparison of
probiotic groups with placebo (post hoc ANOVA: Dunnet t-test)

| | Bacillus | Probiotic |
|---|---|---|
| | coagulans | cocktail |
| | (N* = 25) | (N* = 25) |
| HDLchange [mmol/L] | | |
| mean difference vs placebo | 0.00 (−0.12; | 0.00 (−0.12; |
| (95% CI) | 0.11) | 0.11) |
| p-value vs placebo | 1.000 | 1.000 |
| LDLchange [mmol/L] | | |
| mean difference vs placebo | −0.05 (−0.34; | −0.08 (−0.37; |
| (95% CI) | 0.23) | 0.21) |
| p-value vs placebo | 0.972 | 0.888 |
| TCchange [mmol/L] | | |
| mean difference vs placebo | 0.01 (−0.41; | −0.01 (−0.43; |
| (95% CI) | 0.43) | 0.41) |
| p-value vs placebo | 1.000 | 1.000 |
| TGchange [mmol/L] | | |
| mean difference vs placebo | −0.17 (−0.62; | −0.03 (−0.48; |
| (95% CI) | 0.28) | 0.42) |
| p-value vs placebo | 0.762 | 0.999 |

N* = number of participants included in ITT population

Mean differences with 95% confidence intervals and p-values of pairwise comparison between individual probiotic group and placebo group are shown in Table 20. Compared to placebo, none of the study products containing probiotics showed a statistically significant difference in HDL, LDL, TC or TG relative change from baseline to the end of treatment period.

TABLE 20
Cholesterol and triglyceride relative change from baseline to the
end of the treatment period in ITT population: Pairwise comparison
of probiotic groups with placebo (post hoc ANOVA: Dunnet t-test)

| | Bacillus | Probiotic |
|---|---|---|
| | coagulans | cocktail |
| | (N* = 25) | (N* = 25) |
| HDL relative change [%] | | |
| mean difference vs placebo | −0.56 (−7.87; | −0.58 (−7.89; |
| (95% CI) | 6.76) | 6.74) |
| p-value vs placebo | 0.999 | 0.999 |
| LDLrelative change [%] | | |
| mean difference vs placebo | −2.45 (−12.19; | −4.37 (−14.12; |
| (95% CI) | 7.30) | 8.62) |
| p-value vs placebo | 0.926 | 0.632 |
| TCrelative change [%] | | |
| mean difference vs placebo | 0.87 (−6.37; | −0.40 (−7.64; |
| (95% CI) | 8.11) | 6.84) |
| p-value vs placebo | 0.995 | 1.000 |
| TGrelative change [%] | | |
| mean difference vs placebo | −5.70 (−35.29; | 6.40 (−23.18; |
| (95% CI) | 23.89) | 35.99) |
| p-value vs placebo | 0.970 | 0.955 |

N* = number of participants included in ITT population,
*p-value of Dunnet T-test (pairwise comparison)

Table 21 represents proportion of people who had reported at least one day with clinically relevant infection in Participant diary 1. In Table 22 descriptive statistics of number of days with clinically relevant infection and results of the Kruskal-Wallis test (p*-value) and Mann-Whitney U test with Holm's correction (p-value) are presented.

TABLE 21
Proportion of people with clinically relevant infection
reported in Participant diary 1 (N = 123)

| | Bacillus | Probiotic | |
|---|---|---|---|
| | coagulans | cocktail | Placebo |
| | (N* = 25) | (N* = 25) | (N* = 24) |
| Gastrointestinal | 0/25 (0.0) | 0/25 (0.0) | 2/24 (8.3) |
| infection (%) | | | |
| Respiratory tract | 1/25 (4.0) | 3/25 (12.0) | 6/24 (25.0) |
| infection (%) | | | |
| Urinary tract | 1/25 (4.0) | 2/25 (8.0) | 1/24 (4.2) |
| infection (%) | | | |

N* = number of participants included in the ITT population

TABLE 22
Number of days with clinically relevant infection
reported in Participant diary 1 (N = 123)

| | Bacillus | Probiotic | |
|---|---|---|---|
| | coagulans | cocktail | Placebo |
| | (N* = 25) | (N* = 25) | (N* = 24) |
| Gastrointestinal | 25 | 25 | 24 |
| infection | | | |
| (p* = 0.081) | | | |
| mean (SD): | 0.0 (NC): | 0.0 (NC): | 0.1 (0.4): |
| min-max | 0-0 | 0-0 | 0-2 |
| p-value vs placebo | 0.434 | 0.434 | / |
| Respiratory tract | 25 | 25 | 24 |
| infection | | | |
| (p* = 0.212) | | | |
| mean (SD): | 0.1 (0.6): | 0.6 (2.2): | 1.2 (2.4) |
| min-max | 0-3 | 0-11 | 0-8 |
| p-value vs placebo | 0.142 | 0.236 | / |
| Urinary tract | 25 | 25 | 24 |
| infection | | | |
| (p* = 0.754) | | | |
| mean (SD): | 0.1 (0.6): | 0.2 (0.6): | 0.2 (0.8): |
| min-max | 0-3 | 0-2 | 0-4 |
| p-value vs placebo | 1.000 | 1.000 | / |

N* = number of participants included in the ITT population,
NC = not calculable,
p* = p-value for Kruskal-Wallis test,
p = p-value for Mann-Whitney U test.

Kruskal-Wallis test did not show any significant differences in the number of days with clinically relevant infection treatment groups. However, a borderline statistically significant result was observed for clinically relevant gastrointestinal infection. This is probably due the fact that only no participants in the four probiotic treatment groups experienced clinically relevant gastrointestinal infection, while in probiotic group in total 2 days of such infection were observed, which could have happened by chance.

Nevertheless, compared to placebo, none of the study products containing probiotics showed a statistically significant difference.

Table 23 represents proportion of people who had reported at least one day with individual symptom of gastrointestinal infection in Participant diary 2. In Table 24 descriptive statistics of number of days with symptoms of gastrointestinal infection and results of the Kruskal-Wallis test (p*-value) and Mann-Whitney U test with Holm's correction (p-value) are presented.

TABLE 23
Proportion of people that had reported symptoms of gastrointestinal
infection in Participant diary 2 (N = 118)

| | Bacillus | Probiotic | |
|---|---|---|---|
| | coagulans | cocktail | Placebo |
| | (N* = 25) | (N* = 25) | (N* = 24) |
| Loss of appetite (%) | 0/24 (0.0) | 0/25 (0.0) | 1/23 (4.3) |
| Diarrhea (%) | 0/24 (0.0) | 0/25 (0.0) | 1/23 (4.3) |
| Constipation (%) | 0/24 (0.0) | 0/25 (0.0) | 3/23 (13.0) |
| Vomiting (%) | 0/24 (0.0) | 0/25 (0.0) | 1/23 (4.3) |
| Gases (%) | 0/24 (0.0) | 2/25 (8.0) | 2/23 (8.7) |
| Bowel sounds (%) | 0/24 (0.0) | 0/25 (0.0) | 0/23 (0.0) |
| Cramping/stomach | 1/24 (4.2) | 1/25 (4.0) | 2/23 (8.7) |
| pain (%) | | | |
| Bloating (%) | 1/24 (4.2) | 0/25 (0.0) | 1/23 (4.3) |

N* = number of participants included in the ITT population

TABLE 24
Number of days with symptoms of gastrointestinal infection
reported in Participant diary 2 (N = 118).

| | Bacillus | Probiotic | |
|---|---|---|---|
| | coagulans | cocktail | Placebo |
| | (N* = 25) | (N* = 25) | (N* = 24) |
| Loss of appetite | 24 | 25 | 23 |
| (p* = 0.389) | | | |
| mean (SD): | 0.0 (NC): | 0.0 (NC): | 0.0 (0.2): |
| min-max | 0-0 | 0-0 | 0-1 |
| p-value vs placebo | 0.921 | 1.000 | / |
| Diarrhea | 24 | 25 | 23 |
| (p* = 0.705) | | | |
| mean (SD): | 0.0 (NC): | 0.0 (NC): | 0.0 (0.2): |
| min-max | 0-0 | 0-0 | 0-1 |
| p-value vs placebo | 0.921 | 1.000 | / |
| Constipation | 24 | 25 | 23 |
| (p* = 0.013) | | | |
| mean (SD): | 0.0 (NC): | 0.0 (NC): | 0.4 (1.5): |
| min-max | 0-0 | 0-0 | 0-7 |
| p-value vs placebo | 0.212 | 0.260 | / |
| Vomiting | 24 | 25 | 23 |
| (p* = 0.389) | | | |
| mean (SD): | 0.0 (NC): | 0.0 (NC): | 0.0 (0.2): |
| min-max | 0-0 | 0-0 | 0-1 |
| p-value vs placebo | 0.921 | 1.000 | / |
| Gases | 24 | 25 | 23 |
| (p* = 0.660) | | | |
| mean (SD): | 0.0 (NC): | 0.1 (0.3): | 0.3 (1.3): |
| min-max | 0-0 | 0-1 | 0-6 |
| p-value vs placebo | 0.577 | 0.863 | / |
| Bowel sounds | 24 | 25 | 23 |
| (p* = 1.000) | | | |
| mean (SD): | 0.0 (NC): | 0.0 (NC): | 0.0 (NC): |
| min-max | 0-0 | 0-0 | 0-0 |
| p-value vs placebo | 1.000 | 1.000 | / |
| Cramping/stomach pain | 24 | 25 | 23 |
| (p* = 0.705) | | | |
| mean (SD): | 0.1(0.6): | 0.0 (0.2): | 0.2 (0.9): |
| min-max | 0-3 | 0-1 | 0-4 |
| p-value vs placebo | 1.000 | 1.000 | / |
| Bloating | 24 | 25 | 23 |
| (p* = 0.718) | | | |
| mean (SD): | 0.1 (0.6): | 0.0 (NC): | 0.6 (2.7): |
| min-max | 0-3 | 0-0 | 0-13 |
| p-value vs placebo | 1.000 | 1.000 | / |

N* = number of participants included in the ITT population,
NC = not calculable,
p* = p-value for Kruskal-Wallis test,
p = p-value for Mann-Whitney U test.

A significant difference among groups was detected only in the number of days with constipation (p*=0.013) probably due the fact that only three participants in Placebo group had reported this symptom in Participant diary 2, while in other four treatment groups none of the participants had reported this symptom. However, further analysis (Mann-Whitney U test with Holm's correction) where number of days with constipation was compared between individual probiotic group and placebo group did not show significant differences, probably due to low sample size.

### Efficacy Outcomes: Treatment Period

No significant differences in gastrointestinal health of the participants at baseline among the treatment groups were detected. Participants were randomized to 5 treatment group therefore such results were expected.

Scores of items in Gut-brain axis questionnaire were lower at the end of the treatment period compared to baseline scores, meaning that the participants were less bothered by these symptoms at the end of the treatment period. This can be observed for all treatment groups including the placebo group.

No significant differences in change (end of treatment period minus baseline) of Gut-brain axis item scores among the treatment groups were detected. Compared to placebo group, none of the study products containing probiotics showed a statistically significant difference Gut-brain axis score change.

No significant differences in improvement of Gut-brain axis item scores among the treatment groups were detected. Compared to placebo group, none of the study products containing probiotics showed a statistically significant difference Gut-brain axis score improvement.

No significant differences in sum of scores of all items in Gut brain axis questionnaire among treatment groups were detected. Compared to placebo, none of the study products containing probiotics showed a statistically significant difference in the sum of Gut-brain axis items scores at baseline or at the end of treatment period.

No significant differences in cholesterol and triglyceride changes (absolute and relative change from baseline to the end of the treatment period) among treatment groups were detected.

Compared to placebo, none of the study products containing probiotics showed a statistically significant difference in HDL, LDL, TC or TG change (absolute and relative) from baseline to the end of treatment period. The same findings were observed in the PP population.

Cytokine (TNFα, IFNα, IFNβ, IFNγ, and IL6) levels at the end of the treatment period were below the limit of quantification (LOQ) in all treatment groups. For cytokine IL13 some values above LOQ were reported, but no statistically significant difference between the treatment groups was observed.

No significant changes in heavy metals (Cadmium, Lead, Mercury, Copper, Nickel, Zinc, and Arsenic) levels at the end of the treatment period compared to the baseline values were detected in the Bacillus megaterium group. The same findings were observed in the PP population.

Compared to placebo, none of the study products containing probiotics showed a statistically significant difference in the number of days with symptoms of gastrointestinal infection. However, a borderline statistically significant result was observed for clinically relevant gastrointestinal infection. This is probably due the fact that only no participants in the four probiotic treatment groups experienced clinically relevant gastrointestinal infection, while in probiotic group in total 2 days of such infection were observed, which could have happened by chance.

Compared to placebo, none of the study products containing probiotics showed a statistically significant difference in the number of days with symptoms of respiratory tract infection. However, a statistically significant difference among treatment groups was detected in the number of days with runny nose—thick (p=0.018). probably due the fact that only three participants in Probiotic cocktail group have reported this symptom, while in other four treatment groups none of the participants have reported this symptom.

However, further post hoc analysis (pairwise comparison vs. placebo) did not show any significant differences.

Compared to placebo, none of the study products containing probiotics showed a statistically significant difference in the number of days with symptoms of urinary infection.

No statistically significant differences were detected among the treatment groups in stool regularity.

A significant difference among groups was detected for stool consistency, that is in the proportion of loose stools in the total treatment period as well as in weeks 6 and 7 of the treatment period. However, further post hoc analysis (pairwise comparison vs. placebo) did not show any significant differences, probably due to the low sample size. The participants in the Probiotic cocktail group had the smallest proportion of loose stool per all stools.

### Efficacy Outcomes: Follow-Up Period

A significant difference among groups was detected only in the number of days with constipation (p=0.013). However, further post hoc analysis (pairwise comparison vs. placebo) did not show any significant differences, probably due to low sample size.

None of the study products containing probiotics showed a statistically significant difference in the number of days with respiratory tract infection symptoms.

None of the study products containing probiotics showed a statistically significant difference in the number of days with urinary tract infection symptoms.

No significant differences were detected among the treatment groups in stool regularity and stool consistency.

### Safety Outcomes

In total 17 AEs were reported. Between 1 and 5 ADE were reported for each study product, most commonly gastroesophageal reflux (3 AEs), rash (2 AEs), and vertigo (2 AEs). No SAEs were reported.

Causality assessment revealed no relation between the reported AEs and the study products.

In summary, this study has addressed the safety and efficacy of new probiotics, namely Bacillus coagulans, Bacillus clausii, Bacillus megaterium and probiotic cocktail containing Bacillus subtilis, Bacillus megaterium, Bacillus clausii and Bacillus coagulans.

The gastrointestinal health of the participants at baseline among the treatment groups did not differ between the study groups, which was expected due to randomization.

The primary outcome of the study (safety) was achieved, as 17 AEs were reported in total with no SAEs. Causality assessment revealed no relation between the reported AEs and the study products.

None of the efficacy related outcomes showed any statistically significant difference, however this comes as no surprise due to small sample size per study group. Still, some trends favouring active products were observed, specifically in the Gut-brain axis scores and proportion of loose stools.

To conclude probiotic products showed to be safe to use in adults, and have shown some favourable trends regarding Gut-brain axis and stool consistency, however further studies with larger sample size, a run-in period to determine baseline stool consistency for each participant and use of validated questionnaires to determine the Gut-brain axis domains are needed in order to scientifically prove the efficacy of the studied products.

### REFERENCES

Abriouel, H., Franz, C., Omar, N., & Galvez, A. (2011). Diversity and applications of Bacillus bacteriocins. FEMS Microbiology Reviews, 35(1), 201-232. doi: doi.org/10.1111/j.1574-6976.2010.00244.x
Afrilasari W, Widanarni, Meryadini A (2016) “Effect of Probiotic Bacillus Megaterium PTB 1.4 on the Population of Intestinal Microflora, Digestive Enzyme Activity and the Growth of Catfish (Clarias Sp.).” HAYATI Journal of Biosciences, vol. 23, no. 4, pp. 168-172, doi:10.1016/j.hjb.2016.12.005.
Alippi, A. (1995). Detection of Bacillus larvae spores in Argentinian honeys by using a semi-selective medium. Microbiology SEM, 11, 343-350.
Alippi A, Reynaldi F, Lopez A, De Giusti M, Aguilar O (2004) “Molecular Epidemiology of Paenibacillus Larvae Larvae and Incidence of American Foulbrood in Argentinean Honeys from Buenos Aires Province.” Journal of Apicultural Research, vol. 43, no. 3 pp. 135-143, doi:10.1080/00218839.2004.11101124.
Baron, M. (2009). Original Research: A Patented Strain of Bacillus coagulans Increased Immune Response to Viral Challenge. Postgraduate Medicine, 121(2), 114-118. doi: 10.3810/pgm.2009.03.1971
Benno, Y., Suzuki, K., Suzuki, K., Narisawa, K., Bruce, W. R., & Mitsuoka, T. (1986). Comparison of the Fecal Microflora in Rural Japanese and Urban Canadians. Microbiology and Immunology, 30(6), 521-532. doi: 10.1111/j.1348-0421.1986.tb02978.x
Chan, Y. K., Estaki, M., & Gibson, D. L. (2013). Clinical Consequences of Diet-Induced Dysbiosis. Annals of Nutrition and Metabolism, 63(s2), 28-40. doi: 10.1159/000354902
Cutting, S. M. (2011). Bacillus probiotics. Food Microbiology, 28(2), 214-220. doi: 10.1016/j.fm.2010.03.007
Dolin, B. (2009). Effects of a propietary Bacillus coagulans preparation on symptoms of diarrhea-predominant irritable bowel syndrome. Methods and Findings in Experimental and Clinical Pharmacology, 31(10), 655. doi: 10.1358/mf.2009.31.10.1441078
Elshaghabee, F. M. F., Rokana, N., Gulhane, R. D., Sharma, C., & Panwar, H. (2017). Bacillus As Potential Probiotics: Status, Concerns, and Future Perspectives. Frontiers in Microbiology, 8. doi: 10.3389/fmicb.2017.01490
Endres, J., Clewell, A., Jade, K., Farber, T., Hauswirth, J., & Schauss, A. (2009). Safety assessment of a proprietary preparation of a novel Probiotic, Bacillus coagulans, as a food ingredient. Food and Chemical Toxicology, 47(6), 1231-1238. doi: 10.1016/j.fct.2009.02.018
Ferreira, C. M. H., Vilas-Boas, A., Sousa, C. A., Soares, H. M. V. M., & Soares, E. V. (2019). Comparison of five bacterial strains producing siderophores with ability to chelate iron under alkaline conditions. AMB Express, 9(1). doi: 10.1186/s13568-019-0796-3
Gabrielli, M., Lauritano, E. C., Scarpellini, E., Lupascu, A., Ojetti, V., Gasbarrini, G., . . . Gasbarrini, A. (2009). Bacillus clausii as a Treatment of Small Intestinal Bacterial Overgrowth. The American Journal of Gastroenterology, 104(5), 1327-1328. doi: 10.1038/ajg.2009.91
Gepner, Y., Hoffman, J. R., Shemesh, E., Stout, J. R., Church, D. D., Varanoske, A. N., . . . Ostfeld, I. (2017). Combined effect of Bacillus coagulans GBI-30, 6086 and HMB supplementation on muscle integrity and cytokine response during intense military training. Journal of Applied Physiology, 123(1), 11-18. doi: 10.1152/japplphysiol.01116.2016
Gilliam, Martha. (1979) “Microbiology Of Pollen And Bee Bread: The Genus Bacillus.” Apidologie, vol. 10, no. 3, pp. 269-274, doi:10.1051/apido:19790304.
Gilliam, Martha, and Diane K. Valentine. (1976) “Bacteria Isolated from the Intestinal Contents of Foraging Worker Honey Bees, Apis Mellifera: The Genus Bacillus.” Journal of Invertebrate Pathology, vol. 28, no. 2, pp. 275-276, doi:10.1016/0022-2011(76)90137-3.
Horosheva, T. V., Vodyanoy, V., & Sorokulova, I. (2014). Efficacy of Bacillus probiotics in prevention of antibiotic-associated diarrhoea: a randomized, double-blind, placebo-controlled clinical trial. JMM Case Reports, 1(3). doi: 10.1099/jmmcr.0.004036
Hun, L. (2009). Original Research: Bacillus coagulans Significantly Improved Abdominal Pain and Bloating in Patients with IBS. Postgraduate Medicine, 121(2), 119-124. doi: 10.3810/pgm.2009.03.1984
Hyronimus, B., Marrec, C. L., Sassi, A. H., & Deschamps, A. (2000). Acid and bile tolerance of spore-forming lactic acid bacteria. International Journal of Food Microbiology, 61(2-3), 193-197. doi: 10.1016/s0168-1605(00)00366-4
Ivanovics, G. & Alfoldi, L. (1957). Bacteriocinogenesis in Bacillus megaterium. J. gen. Microbiol. 16, 522
Jensen, G., Cash, H., Farmer, S., & Keller, D. (2017). Inactivated probiotic Bacillus coagulans GBI-30 induces complex immune activating, anti-inflammatory, and regenerative markers in vitro. Journal of Inflammation Research, Volume 10, 107-117. doi: 10.2147/jir.s141660
Kalman, D. S., Schwartz, H. I., Alvarez, P., Feldman, S., Pezzullo, J. C., & Krieger, D. R. (2009). A prospective, randomized, double-blind, placebo-controlled parallel-group dual site trial to evaluate the effects of a Bacillus coagulans-based product on functional intestinal gas symptoms. BMC Gastroenterology, 9(1). doi: 10.1186/1471-230x-9-85
Kawarizadeh, A., Nojoomi, F., Tabatabaei, M., Hosseinzadeh, S., & Farzaneh, M. (2019). The effect of Bacillus coagulans on cytotoxicity and apoptosis induced by Salmonella typhimurium in HT-29 cell culture. Iranian Journal of Microbiology. doi: 10.18502/ijm.v11i4.1468
Keller, D., Dinter, R. V., Cash, H., Farmer, S., & Venema, K. (2017). Bacillus coagulans GBI-30, 6086 increases plant protein digestion in a dynamic, computer-controlled in vitro model of the small intestine (TIM-1). Beneficial Microbes, 8(3), 491-496. doi: 10.3920/bm2016.0196
Khochamit, N., Siripornadulsil, S., Sukon, P., & Siripornadulsil, W. (2015). Antibacterial activity and genotypic-phenotypic characteristics of bacteriocin-producing Bacillus subtilis KKU213: Potential as a probiotic strain. Microbiological Research, 170, 36-50. doi: 10.1016/j.micres.2014.09.004
Kimmel, M., Keller, D., Farmer, S., & Warrino, D. (2010). A controlled clinical trial to evaluate the effect of GanedenBC30 on immunological markers. Methods and Findings in Experimental and Clinical Pharmacology, 32(2), 129. doi: 10.1358/mf.2010.32.2.1423881
Kotb, E. (2014). Purification and partial characterization of serine fibrinolytic enzyme from Bacillus megaterium KSK-07 isolated from kishk, a traditional Egyptian fermented food. Applied Biochemistry and Microbiology, 51(1), 34-43. doi:10.1134/s000368381501007x
Lakshmi, S. G., Jayanthi, N., Saravanan, M., & Ratna, M. S. (2017). Safety assessment of Bacillus clausii UBBC07, a spore forming probiotic. Toxicology Reports, 4, 62-71. doi: 10.1016/j.toxrep.2016.12.004
Liu, P., Xie, J., Liu, J., & Ouyang, J. (2019). A novel thermostable β-galactosidase from Bacillus coagulans with excellent hydrolysis ability for lactose in whey. Journal of Dairy Science, 102(11), 9740-9748. doi: 10.3168/jds.2019-16654
Marseglia, G. L., Tosca, M., Cirillo, I., Licari, A., Leone, M., Marseglia, A., . . . Ciprandi, G. (2007). Efficacy of Bacillus clausii spores in the prevention of recurrent respiratory infections in children: a pilot study. Therapeutics and Clinical Risk Management, 3(1), 13-17. doi: 10.2147/tcrm.2007.3.1.13
Mazzoli, A., Donadio, G., Lanzilli, M., Saggese, A., Guarino, A. M., Rivetti, M., . . . Isticato, R. (2019). Bacillus megaterium SF185 spores exert protective effects against oxidative stress in vivo and in vitro. Scientific Reports, 9(1). doi: 10.1038/s41598-019-48531-4
Neag, M. A., Catinean, A., Muntean, D. M., Pop, M. R., Bocsan, C. I., Botan, E. C., & Buzoianu, A. D. (2020). Probiotic Bacillus Spores Protect Against Acetaminophen Induced Acute Liver Injury in Rats. Nutrients, 12(3), 632. doi: 10.3390/nu12030632
Nista, E. C., Candelli, M., Cremonini, F., Cazzato, I. A., Zocco, M. A., Franceschi, F., . . . Gasbarrini, A. (2004). Bacillus clausii therapy to reduce side-effects of anti-Helicobacter pylori treatment: randomized, double-blind, placebo controlled trial. Alimentary Pharmacology and Therapeutics, 20(10), 1181-1188. doi: 10.1111/j.1365-2036.2004.02274.x
Nithya, V., & Halami, P. M. (2012). Evaluation of the probiotic characteristics of Bacillus species isolated from different food sources. Annals of Microbiology, 63(1), 129-137. doi:10.1007/s13213-012-0453-4
Padgham, J., and R. Sikora. (2007) “Biological Control Potential and Modes of Action of Bacillus megaterium against Meloidogyne Graminicola on Rice.” Crop Protection, vol. 26, no. 7, pp. 971-977, doi:10.1016/j.cropro.2006.09.004.
Pelletier, A. & Sygush, J (1990). Purification and characterization of three chitosanase activities from Bacillus megaterium P1. Appl Environ Microbiol 56, 844-848
Ripert, G., Racedo, S. M., Elie, A.-M., Jacquot, C., Bressollier, P., & Urdaci, M. C. (2016). Secreted Compounds of the Probiotic Bacillus clausii Strain O/C Inhibit the Cytotoxic Effects Induced by Clostridium difficile and Bacillus cereus Toxins. Antimicrobial Agents and Chemotherapy, 60(6), 3445-3454. doi: 10.1128/aac.02815-15
Rochín-Medina, J. J., Ramirez-Medina, H. K., Rangel-Peraza, J. G., Pineda-Hidalgo, K. V., & Iribe-Arellano, P. (2017). Use of whey as a culture medium for Bacillus clausii for the production of protein hydrolysates with antimicrobial and antioxidant activity. Food Science and Technology International, 24(1), 35-42. doi: 10.1177/1082013217724705
Salvetti, E., Orrh, L., Capozzi, V, Martina Am Lamontanara A, Keller D, Cash H, Felis G, Cattivelli L, Torriani S, Spano G. (2016) Integrate genome-based assessment of safety for probiotic strains: Bacillus coagulans GBI-30, 6086 as a case study. Appl Microbiol Biotechnol 100: 4595. https://doi.org/10.1007/s00253-016-7416-9
Scholle M D, White C A, Kunnimalaiyaan M, Vary P S (2003) “Sequencing and Characterization of pBM400 from Bacillus megaterium QM B1551.” Applied and Environmental Microbiology, vol. 69, no. 11, pp. 6888-6898, doi:10.1128/aem.69.11.6888-6898.2003.
Sharma, S., & Kanwar, S. S. (2017). Adherence potential of indigenous lactic acid bacterial isolates obtained from fermented foods of Western Himalayas to intestinal epithelial Caco-2 and HT-29 cell lines. Journal of Food Science and Technology, 54(11), 3504-3511. doi: 10.1007/s13197-017-2807-1
Snowdon, Jill A, and Dean O Cliver. (1996) “Microorganisms with Honey.” International Journal of Food Microbiology.
Sudha, M., Radkar, N., & Maurya, A. (2012). Effect of Supplementation of Probiotic Bacillus coagulans Unique IS-2 on Hypercholesterolemia Subjects: A Clinical Study. International Journal of Probiotics and Prebiotics, 6(2).
Sudha, M. R., Yelikar, K. A., & Deshpande, S. (2012). Clinical Study of Bacillus coagulans Unique IS-2 (ATCC PTA-11748) in the Treatment of Patients with Bacterial Vaginosis. Indian Journal of Microbiology, 52(3), 396-399. doi: 10.1007/s12088-011-0233-z
Sudha, M. R., Bhonagiri, S., & Kumar, M. A. (2013). Efficacy of Bacillus clausii strain UBBC-07 in the treatment of patients suffering from acute diarrhoea. Beneficial Microbes, 4(2), 211-216. doi: 10.3920/bm2012.0034
Sudha, M. R., Jayanthi, N., Aasin, M., Dhanashri, R., & Anirudh, T. (2018). Efficacy of Bacillus coagulans Unique IS2 in treatment of irritable bowel syndrome in children: a double blind, randomised placebo controlled study. Beneficial Microbes, 9(4), 563-572. doi: 10.3920/bm2017.0129
Sudha, M. R., Jayanthi, N., Pandey, D., & Verma, A. (2019). Bacillus clausii UBBC-07 reduces severity of diarrhoea in children under 5 years of age: a double blind placebo controlled study. Beneficial Microbes, 10(2), 149-154. doi: 10.3920/bm2018.0094
Sumathi C, Nandhini A, Padmanaban J (2017) “Antagonistic Activity of Probiotic Bacillus megaterium against Streptococcus mutans.” International Journal of Pharma and Bio Science, vol. 8, no. 1, doi:10.22376/ijpbs.2017.8.1.p270-274.
Thakur, K., Tomar, S. K., & De, S. (2016). Lactic acid bacteria as a cell factory for riboflavin production. Microbial Biotechnology, 9(4), 441-451. doi: 10.1111/1751-7915.12335
Tysset, C., Durand, C., and Taliergio, Y. P. (1970) Contribution to the study of the microbial contamination and the hygiene of commercial honey. Rec. Med. Vet. 146, 1471-1492.
Vary, P. S., Biedendieck, R., Fuerch, T., Meinhardt, F., Rohde, M., Deckwer, W., & Jahn, D. (2007). Bacillus megaterium—from simple soil bacterium to industrial protein production host. Applied Microbiology and Biotechnology, 76(5), 957-967. doi:10.1007/s00253-007-1089-3
von Tersch, M. A. and Carlton, B. C. (1983) Bacteriocin from Bacillus megaterium ATCC 19213: comparative studies with megacin A-216. Journal of Bacteriology 155, 866-871.

The invention is not limited to the embodiment described herein but can be amended or modified without departing from the scope of the present invention.

The use of the terms “a,” “an,” “the,” and similar referents in the context of describing the present invention (especially in the context of the claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Use of the term “about” is intended to describe values either above or below the stated value in a range of approximately ±10%; in other embodiments, the values may range in value above or below the stated value in a range of approximately ±5%; in other embodiments, the values may range in value above or below the stated value in a range of approximately ±2%; in other embodiments, the values may range in value above or below the stated value in a range of approximately ±1%. The preceding ranges are intended to be made clear by context, and no further limitation is implied. All methods described herein can be performed in any suitable order unless otherwise indicated here in or otherwise clearly contradicted by context. The use of any and all examples, or exemplary language (e.g., “such as”) provided herein, is intended merely to better illuminate the invention and does not pose a limitation on the scope of the invention unless otherwise stated. No language in the specification should be construed as indicating any non-claimed element as essential to the practice of the invention.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entireties. The present invention may be embodied in other specific forms without departing from the spirit or essential attributes thereof, and, accordingly, reference should be made to the appended claims, rather than to the foregoing specification, as indicating the scope of the invention.

1. A Bacillus coagulans strain comprising a purified microbial population that comprises one or more bacteria with a gyrB that shares at least 97% identity with SEQ ID NO: 1; and/or that comprises one or more bacteria with a 16S rRNA that shares at least 97% identity with SEQ ID NO: 2.

2. The Bacillus coagulans strain of claim 1 that shares at least 97% identity with SEQ ID NO: 3.

3. The Bacillus coagulans strain of claim 1 wherein the purified microbial population comprises a bacterium with a 16S nucleic acid sequence comprising SEQ ID NO:2.

4. The Bacillus coagulans strain of claim 1 wherein the purified microbial population comprises a bacterium with gyrB nucleic acid sequence comprising SEQ ID NO: 1.

5. The Bacillus coagulans strain of claim 1 wherein the purified microbial population comprises a bacterium with a 16S nucleic acid sequence comprising SEQ ID NO: 2 and with a gyrB nucleic acid sequence comprising SEQ ID NO: 1.

6. A microbial composition comprising the Bacillus coagulans strain of claim 1 together with a comestibly acceptable carrier and/or diluent.

7. The microbial composition of claim 6, wherein a unit dose of the composition comprises 10⁶-10¹³ CFU of the Bacillus coagulans strain.

8. The microbial composition of claim 6, further comprising a mucous adherent excipient.

9. The microbial composition of claim 6, further comprising at least one further probiotic Bacillus spp. strain.

10. The microbial composition of claim 6, wherein the microbial composition is formulated as a tablet, a pill, a capsule, a powder, a solution, a suspension, or an emulsion.

11. The microbial composition of claim 6, wherein the microbial composition is formulated as a food.

12. (canceled)

13. A method of treating vaginal infections, urinary tract infections, gastrointestinal infections, gastrointestinal diseases, improving immune health, protection against oxidative stress, cleansing and detoxification, improving metabolic health or cardiovascular health, the method comprising the step of administering to a human subject the Bacillus coagulans strain of claim 1.

14. (canceled)

15. A method of treating vaginal infections, urinary tract infections, gastrointestinal infections, gastrointestinal diseases, improving immune health, protection against oxidative stress, cleansing and detoxification, improving metabolic health or cardiovascular health, the method comprising the step of administering to a human subject the microbial composition of claim 6.

16. A method of improving microbiome within a subject, comprising the step of administering to the subject a composition comprising a probiotic, wherein the probiotic comprises the Bacillus coagulans strain of claim 1.

17. (canceled)

18. (canceled)
